(19) United States
(12) Patent Application Publication
(10) Pub. No.: US 2025/0002862 A1
(43) Pub. Date: Jan. 2, 2025

(54) Manufacturing of Bioengineered Recellularized Organs and Methods of Use Thereof

(71) Applicant: Miromatrix Medical Inc., Eden Prairie, MN (US)

(72) Inventors: Jeff J. ROSS, Eden Prairie, MN (US); Christopher J. FECTEAU, Eden Prairie, MN (US); John J. BARRY, Eden Prairie, MN (US); Shawn RIESGRAF, Eden Prairie, MN (US); Isaac BERGSTROM, Eden Prairie, MN (US)

(21) Appl. No.: 18/759,375

(22) Filed: Jun. 28, 2024

Related U.S. Application Data
(60) Provisional application No. 63/511,413, filed on Jun. 30, 2023.

(51) Int. Cl. C12N 5/071

(52) U.S. Cl. CPC C12N 5/0671; C12N 2509/10; C12N 2513/00; C12N 2533/90

(57) ABSTRACT

Provided herein are methods and compositions relating to at least partially recellularized human organs. Various methods of decellularizing non-human animal organs and recellularizing a non-human animal extracellular matrix with cell compositions. Further provided are compositions and methods for treating a liver or other disease (such as acute liver failure) using an extracorporeal bioengineered liver or other organ.

### CROSS-REFERENCE TO RELATED APPLICATIONS

This application claims priority to, and the benefit of, U.S. Provisional Application No. 63/511,413, filed Jun. 30, 2023, the contents of which is entirely incorporated herein by reference for all purposes and commonly owned.

### BACKGROUND

Organ transplants are common across the United States and throughout the world. Organs such as livers, kidneys, pancreas, lungs and hearts are commonly transplanted to prolong the life of the recipients. However, there is often a shortage of organs, creating a demand and a waiting list for the organs. As one example, Acute Liver Failure (ALF), can be a life-threatening, critical illness in patients that is characterized by the rapid onset of abnormal liver biochemistry, coagulopathy, and often the progression to encephalopathy. While clinical outcomes for patients with ALF have steadily improved with changes in medical management, the gold standard to treat ALF remains liver transplantation, with a 1-year survival rate of 91%. Data from the Acute Liver Failure Study Group (ALFSG) Registry and the Scientific Registry of Transplant Recipients (SRTR) reported that only 64% of patients listed for transplant received a lifesaving organ. Strategies are needed to mitigate the long waitlists for organ transplants and improve patient quality of life.

### SUMMARY

Provided herein are bioengineered, recellularized organs for use in the treatment of various diseases. In some embodiments, the bioengineered, recellularized organ comprises a liver or a liver has reduced and/or inactivated microbial particles, can be cultured for long periods of time (e.g., greater than 4 days) prior to transplantation or use in an ex-vivo blood circuit without systemic anticoagulation, and can be stored in cold storage for more than 6 hours and maintain liver function (e.g., ammonia clearance and urea production). Further provided herein are methods of manufacturing bioengineered liver tissues and methods of treating a patient with an extracorporeal liver.

Provided herein are methods of making an at least partially recellularized organ composition, including but not limited to liver, kidney, lung and heart, wherein the methods comprise: (a) treating a non-human animal organ with an anti-viral treatment; (b) perfusion decellularizing the non-human animal organ to obtain a decellularized extracellular matrix; (c) contacting the decellularized extracellular matrix with a cell composition comprising a population of human cells to form an at least partially recellularized organ composition.

Provided herein are methods of making an at least partially recellularized liver composition, wherein the methods comprise: (a) treating a non-human animal liver with an anti-viral treatment; (b) perfusion decellularizing the non-human animal liver to obtain a decellularized extracellular matrix; (c) contacting the decellularized extracellular matrix with a cell composition comprising a population of human liver cells to form an at least partially recellularized liver composition.

Provided herein are methods of making an at least partially recellularized organ composition, wherein the methods comprise: (a) treating a non-human animal organ with an anti-viral treatment; (b) perfusion decellularizing the non-human animal organ to obtain a decellularized extracellular matrix; (c) contacting the decellularized extracellular matrix with a cell composition comprising a population of human cells to form an at least partially recellularized organ composition.

Provided herein are methods of making an at least partially recellularized organ composition, the methods comprising: (a) treating a non-human animal organ with an anti-viral treatment; (b) perfusion decellularizing the non-human animal organ to obtain a decellularized extracellular matrix; (c) contacting the decellularized extracellular matrix with a first cell composition comprising a population of human vascular endothelial cells; and (d) contacting the decellularized extracellular matrix with a second cell composition to form an at least partially recellularized organ composition.

Provided herein are methods of making an at least partially recellularized liver composition, the methods comprising: (a) treating a non-human animal liver with an anti-viral treatment; (b) perfusion decellularizing the non-human animal liver to obtain a decellularized extracellular matrix; (c) contacting the decellularized extracellular matrix with a first cell composition comprising a population of human vascular endothelial cells; and (d) contacting the decellularized extracellular matrix with a second cell composition comprising a population of human liver cells to form an at least partially recellularized liver composition.

Provided herein are compositions comprising an at least partially recellularized organ produce by any of the methods provided herein.

Provided herein are compositions comprising an at least partially recellularized liver composition produced by any of the methods provided herein.

Provided herein are at least partially recellularized livers comprising: (a) a porcine extracellular matrix; (b) a population of human endothelial cells and a population of human liver cells engrafted onto the porcine extracellular matrix, wherein the at least partially recellularized livers have an increase in ammonia clearance relative to a population of porcine liver cells engrafted onto a porcine extracellular matrix.

Provided herein are at least partially recellularized livers comprising: (a) a microbial particle diminished, perfusion-decellularized porcine extracellular matrix; (b) a population of human endothelial cells and a population of human liver cells engrafted onto the porcine extracellular matrix, wherein the at least partially recellularized livers have an increase in ammonia clearance relative to a population of porcine liver cells engrafted onto a porcine extracellular matrix.

Provided herein are compositions, wherein the compositions comprise: the at least partially recellularized liver provided herein; and an extracellular matrix protein or solution. Further provided herein are compositions, wherein the compositions further comprise cell culture medium.

Provided herein are ex-vivo methods of treating a liver disease in a subject, wherein the methods comprise: producing a blood circuit, wherein the blood circuit comprises blood from the subject in fluid communication with an at least partially recellularized liver provided herein, wherein the at least partially recellularized liver filters blood from the subject and clears ammonia, thereby treating the liver disease in the subject.

Provided herein are methods for treating a liver disease in a subject, wherein the methods comprise: administering to a subject the at least partially recellularized liver provided herein, thereby treating the liver disease in the subject. Further provided herein are methods, wherein the liver disease comprises acute liver failure (ALF).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosed embodiments are set forth with particularity in the appended claims. A better understanding of the features and advantages of the disclosed embodiments will be obtained by reference to the following detailed description that sets forth illustrative embodiments, in which the principles of the disclosed embodiments are utilized, and the accompanying drawings of which:

FIG. 1A to FIG. 1C shows a schematic representation of an embodiment of the method of manufacturing a recellularized liver and methods of treating a subject with a liver disease. FIG. 1A shows a schematic representation with photographs in which native porcine livers are harvested, decellularized, and recellularized with HUVECs and human hepatocytes. FIG. 1B shows schematic representation of a bioreactor station used to culture the engineered liver. FIG. 1C shows a cell culture scheme showing the scaffold equilibration and qualification period, endothelial culture of HUVECs, and bi-culture of HUVECs and human hepatocytes, including the 3-day available therapy window.

FIG. 2A to FIG. 2I shows cellular identification and histology of extracorporeal bioengineered livers (BELs). HUVECs and PHLCs were characterized prior to seeding and within the graft. HUVECs expression of CD31 and CD105 was evaluated by flow cytometry prior to seeding into the decellularized graft. FIG. 2A shows a representative histogram of HUVEC expression of endothelial markers. FIG. 2B shows a graph of the percentage of HUVECs stained positive for CD31 and CD105 from FIG. 1A (n=25). FIG. 2C shows a graph of the glucose consumption rate during the endothelial culture period of the decellularized scaffold. The shaded area depicts two standard deviations from the mean. FIG. 2D shows a flow cytometry scatter profile of PHLC population. Hepatocytes are indicated based on size and complexity by gated area. Gating is representative. FIG. 2E shows ASGR expression within a representative PHLC population. Gate indicates positive expression as compared to appropriate isotype control. The population shown is representative of the PHLC population after isolation. FIG. 2F shows hepatocyte quantification within the BEL population as determined from the scatter plot gating strategy. (n=14) FIG. 2G shows hematoxylin and eosin (H&E) staining showing a cross-section of a BEL recellularized with HUVECs and PHLCs one-day post hepatocyte seeding. FIG. 2H shows H&E staining a cross-section of a HUVEC lined vessel in a BEL recellularized with HUVECs and PHLCs. Arrows show representative areas of HUVEC-lined vessels. FIG. 2I shows H&E staining H&E showing a highly magnified cross-section of a BEL recellularized with HUVECs and PHLCs hepatocytes one-day post hepatocyte seeding. Arrows show HUVECs lining the vessel.

FIG. 3A to FIG. 3H shows a comparison of function between porcine and human recellularized livers (BELs). FIG. 3A shows a graph showing that ammonia cleared from media after one hour post 200 μM bolus to the porcine and human BEL, data expressed per million cells (n=23 porcine, n=16 human). FIG. 3B shows a graph showing one hour of ammonia clearance was performed daily for 3 days of BEL culture in porcine and human BELs, with data expressed per million cells (n=5 porcine, 10 human). FIG. 3C shows a graph showing glucose consumption calculated daily after 24 h of culture for 3 days in porcine and human BELs (n=24 porcine, 10 human) FIG. 3D shows a graph showing Urea concentration in BEL conditioned media collected at the end of a 24-h culture period, data expressed per million cells (n=14 porcine, n=8 human). FIG. 3E shows a graph showing Fibrinogen production in BEL conditioned media collected at the end of a 24-h culture period, data expressed per million cells (n=5 porcine, n=8 human) FIG. 3F shows a graph showing A1AT production in BEL conditioned media collected at the end of a 24-h culture period, data expressed per million cells (n=5 porcine, n=8 human). FIG. 3G shows a schematic showing circuit for blood perfusion studies. FIG. 3H shows a graph showing porcine and human BEL patency was measured for 180 min in a blood perfusion circuit (n=6 porcine BEL, 3 human BEL). Statistical significance **p<0.01, ***p<0.001, ****p<0.0001, and ns=not significant. represent the results of a t-test between porcine and human data, and both (FIG. 3B, FIG. 3C) show results with a false discovery correction.

FIG. 4A to FIG. 4F shows Human BELs display extended function following a cold storage period. FIG. 4A shows a schematic diagram showing assay sampling scheme during culture period and 3-day extended hold period during the therapy window after a 14-16 h of cold storage static hold (CS). FIG. 4B shows a graph of ammonia cleared from culture media by human BEL after 1-h post-delivery of 200 μM bolus of ammonia, performed daily during the therapy window after the cold storage hold period (n=5-7). FIG. 4C shows a graph of daily measurements of glucose consumption (n=4-9), FIG. 4D shows a graph of urea production (n=4-7), FIG. 4E shows a graph of fibrinogen (n=4-7), and FIG. 4F shows a graph of AAT (n=4-7, n=2 for measurements collected on day 6 and 7) production were quantified in human BELs each day after 24 h of culture during the post cold storage therapy window. Statistical significance *p<0.05, **p<0.01, ****p<0.0001, and ns=not significant.

FIG. 5A to FIG. 5D shows that human recellularized livers display extended function for at least 7 days. FIG. 5A shows a graph of ammonia clearance over time. FIG. 5B shows a graph of urea concentration over time. FIG. 5C shows a graph of fibrinogen concentration over time. FIG. 5D shows a graph of alpha-1 antitrypsin (A1AT) concentration over time.

FIG. 6A to FIG. 6D shows that human recellularized livers remain functional for at least 4 days after cold storage. FIG. 6A shows a graph of ammonia clearance over time. FIG. 6B shows a graph of urea concentration over time. FIG. 6C shows a graph of fibrinogen concentration over time. FIG. 6D shows a graph of alpha-1 antitrypsin (A1AT) concentration over time. Arrows denote 24 hours after return to normothermic perfusion within bioreactor following 12 hours of a cold storage transport simulation (PT=Post Transport).

FIG. 7 shows an embodiment of the ex-vivo blood circuit.

FIG. 8 shows a photograph showing that E-beam treatment of a perfusion-decellularized porcine liver after decellularization reduces the desired shape and orientation of the extracellular matrix needed for organ recellularization to be performed.

FIG. 9 shows a photograph of harvested, frozen, porcine liver that to be treated with an anti-viral treatment prior to decellularization.

FIG. 10 shows a photograph of a perfusion-decellularized porcine liver when treated with an e-beam prior to decellularization.

FIG. 11 shows a graph of glucose consumption rates for recellularized livers comparing the livers produced using the anti-viral pretreatment step relative to non-pretreated controls.

FIG. 12 shows a graph of blood loop patency recellularized livers produced using the anti-viral pretreatment step relative to non-pretreated controls.

### DETAILED DESCRIPTION OF THE INVENTION

Provided herein are bioengineered, recellularized organs, such as a liver (also referred to herein as an at least partially recellularized liver) for use in the treatment of liver diseases. Provided herein are methods of preparing at least partially recellularized livers. The bioengineered, recellularized livers have reduced and/or inactivated microbial particles, can be cultured for long periods of time (e.g., greater than 4 days) prior to transplantation or use in an ex-vivo blood circuit. The inventors have discovered that the combination of an anti-viral treatment, using a porcine decellularized extracellular matrix, and cell seeding conditions can improve the health and function of a bioengineered recellularized liver that comprises human cells. The inventors also discovered that a bioengineered recellularized liver produced by the methods provided herein can be used ex-vivo to clear ammonia, produce urea, produce fibrinogen, and alpha-1-antitrypsin (A1AT) in a blood circuit of a subject. This function can be maintained for at least 4 days after cold storage and can function for at least 7 days in a bioreactor. Surprisingly and unexpectedly, the claimed methods of making an at least partially recellularized liver has improved liver function when human cells were seeded on a decellularized porcine extracellular matrix relative to recellularization of the decellularized porcine extracellular matrix with porcine liver cells.

Provided herein are methods of making an at least partially recellularized liver composition. Further provided herein are least partially recellularized livers comprising: (a) a decellularized non-human animal extracellular matrix; and (b) a population of human endothelial cells and a population of human liver cells engrafted onto the non-human animal extracellular matrix, wherein the at least partially recellularized liver has an increase in ammonia clearance relative to a population of non-human animal liver cells that are engrafted onto a comparable non-human animal extracellular matrix.

Provided herein are ex-vivo methods of treating a liver disease in a subject, the method comprising: producing a blood circuit, wherein the blood circuit comprises blood from the subject in fluid communication with an at least partially recellularized liver provided herein, wherein the at least partially recellularized liver filters blood from the subject and clears ammonia, thereby treating the liver disease in the subject.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

The term “about” and its grammatical equivalents in relation to a reference numerical value and its grammatical equivalents as used herein may include a range of values plus or minus 10% from that value. For example, the amount “about 10” includes amounts from 9 to 11. The term “about” in relation to a reference numerical value may also include a range of values plus or minus: 10%, 9%, 8%, 7%, 6%, 5%, 4%, 3%, 2%, or 1% from that value.

The term “substantially” as used herein may refer to a value approaching 100% of a given value. In some embodiments, the term may refer to an amount that may be at least about 90%, 91%, 92%, 93%, 94%, 95%, 96%, 97%, 98%, 99%, 99.9%, or 99.99% of a total amount. In some embodiments, the term may refer to an amount that may be about 100% of a total amount.

The term “decellularized” or “decellularization” as used herein may refer to a biostructure (e.g., an isolated organ or portion thereof, or tissue), from which the cellular and tissue content has been reduced or removed leaving behind an intact acellular infra-structure. Organs such as the kidney can be composed of various specialized tissues. Specialized tissue structures of an organ, or parenchyma, can provide specific function associated with the organ. Supporting fibrous network of an isolated organ can be a stroma. Most organs have a stromal framework composed of unspecialized connecting tissue which supports the specialized tissue. The process of decellularization may at least partially remove the cellular portion of the tissue, leaving behind a complex three-dimensional network of extracellular matrix (ECM). An ECM infrastructure may primarily be composed of collagen but can include cytokines, proteoglycans, laminin, fibrillin and other proteins secreted by cells. An at least partially decellularized structure may provide a biocompatible substrate onto which different cell populations may be infused or used to be implanted as acellular medical devices such as but not limited to, wound care matrix, fistula matrix, void filler, dermal fillers, soft tissue reinforcement, or other substrates that enable cellular infiltration and remodeling following implantation or application. Decellularized biostructures may be rigid, or semi-rigid, having an ability to alter their shapes. Examples of decellularized isolated organs may include, but are not limited to solid organs such as, a heart, kidney, liver, lung, pancreas, brain, bone, spleen, gall bladder, urinary bladder, uterus, ureter, and urethra.

The term “recellularize” or “recellularization” as used herein may refer to an engraftment or distribution of a plurality of cells or a cell composition as provided herein onto a decellularized extracellular matrix. A recellularized organ may comprise morphology or activity of a native, non-decellularized organ.

The term “effective amount” or “therapeutically effective amount” may refer to a quantity of a composition, for example a composition comprising cells such as cells, that can be sufficient to result in a desired activity upon introduction into an isolated organ or portion thereof provided herein.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

The term “recipient” and their grammatical equivalents as used herein may refer to a subject. A subject may be a human or non-human animal. A recipient may also be in need thereof, such as needing treatment for a disease such as cancer. In some embodiments, a recipient may be in need thereof of a preventative therapy. A recipient may not be in need thereof in other cases.

The term “subject” and its grammatical equivalents as used herein may refer to a human or a non-human. A subject may be a mammal. A subject may be a human mammal of a male or female biological gender. A subject may be of any age. A subject may be an embryo. A subject may be a newborn or up to about 100 years of age. A subject may be in need thereof. A subject may have a disease such as cancer. A subject may be premenopausal, menopausal, or have induced menopause.

The terms “treatment” or “treating” and their grammatical equivalents may refer to the medical management of a subject with an intent to cure, ameliorate, stabilize, or prevent a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include preventative treatment, that is, treatment directed to minimizing or partially or completely inhibiting the development of a disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure, ameliorate, stabilize or prevent a disease, condition, or disorder.

### Organ Anti-Microbial Treatment

Provided herein are at least partially recellularized organs or portions thereof (e.g., livers) that are free or substantially free of microbial organisms and/or have reduced or inactivated microorganisms. In some instances, a “microbial organism” and a “microbial particle” are used herein interchangeably. In some embodiments, the microbial organism can be a eukaryotic cell or organism, or a prokaryotic cell or organism, a virus, a bacteria, a fungus, a yeast, a protein such as a prion, a parasite, an endotoxin, or any combination thereof. In some embodiments, the at least partially recellularized organs or portions thereof are free of viruses or viral particles. In some embodiments, the anti-microbial treatment comprises an anti-viral treatment.

Prior to decellularizing a non-human animal organ provided herein, the non-human animal organ is removed or isolated from the non-human animal and frozen. In some embodiments, the isolated non-human animal liver is stored at a temperature of about −80 degrees Celsius up to about 0 degrees Celsius. In some embodiments, the isolated non-human animal liver is stored at a temperature of about −20 degrees Celsius up to about −10 degrees Celsius.

The frozen non-human animal organ (e.g., liver) is treated with an anti-viral treatment provided herein. In some embodiments, the anti-viral treatment comprises treating or irradiating the non-human animal organ with radiation. In some embodiments, the anti-viral treatment comprises treating or irradiating the non-human animal organ with an electron beam (E-beam). In irradiation processing, the dose of the electron beam is the amount of energy absorbed by the target material (e.g., the non-human animal liver) which is in the units of Gray, where 1 Gray is equal to 1 Joule per kilogram. In some embodiments, the non-human animal organ is treated with an electron beam at an electron beam dose from about 1 kiloGray (kGy) up to about 100 kGy. In some embodiments, the electron beam dose is from about 2 kGy to about 50 kGy. In some embodiments, the electron beam dose is from about 5 kGy to about 25 kGy. In some embodiments, the electron beam dose is from about 10 kGy to about 20 kGy. In some embodiments, the electron beam dose is from between 10 kGy and 20 kGy. In some embodiments, the non-human animal organ is irradiated by an electron beam at an electron beam dose of at least 5 kGY, at least 6 kGY, at least 7 kGY, at least 8 kGY, at least 9 kGY, at least 10 kGY, at least 11 kGY, at least 12 kGY, at least 13 kGY, at least 14 kGY, at least 15 kGY, at least 16 kGY, at least 17 kGY, at least 18 kGY, at least 19 kGY, at least 20 kGY, at least 21 kGY, at least 22 kGY, at least 23 kGY, at least 24 kGY, at least 25 kGY, or more.

In some embodiments, the non-human animal organ is treated with irradiation for a period of about 10 seconds up to about 200 seconds. In some embodiments, the non-human animal organ is treated with irradiation for a period of at least 30 seconds, at least 1 minute (60 seconds), at least 2 minutes, at least 3 minutes, at least 4 minutes, or at least 5 minutes or more. In some embodiments, the non-human animal organ is treated with irradiation for a period of about 5 minutes up to about 1 hour (60 minutes). In some embodiments, the non-human animal organ is treated with irradiation for a period of about 1 hours up to about 24 hours. some embodiments, the non-human animal organ is treated with irradiation for a period of about 24 hours (1 day) up to about 168 hours (7 days).

In some embodiments, the anti-microbial treatment (e.g. anti-viral treatment) comprises exposing the non-human animal organ to ultraviolet light, plasma, gamma rays, or X-rays. The time in which the non-human animal organ is exposed to an anti-viral treatment provided herein will depend upon the type of treatment being used to remove viruses and viral particles.

In some embodiments, the anti-viral treatment comprises a chemical treatment. In some embodiments, the chemical treatment is in addition to the radiation treatment, for example electron beam treatment. In some embodiments, the chemical treatment comprises contacting the non-human animal organ or the decellularized extracellular matrix of the non-human animal lever with a detergent, an enzyme, an anti-microbial agent, a peroxide, a peroxy acid, or any combination thereof.

Antimicrobial agents can be non-oxidative organic chemicals which derive their antimicrobial activity through a chemical or physicochemical interaction with the microorganisms. Suitable antimicrobial agents are polymeric quaternary ammonium salts, for example, poly[(dimethyliminio)-2-butene-1,4-diyl chloride], [4-tris(2-hydroxyethyl) ammonio]-2-butenyl-w-[tris(2-hydroxyethyl)ammonio]dichloride (chemical registry number 75345-27-6), benzalkonium halides, and biguanides such as salts of alexidine, alexidine free base, salts of chlorhexidine, hexamethylene biguanides and their polymers.

Proteolytic enzymes can be used in combination with the anti-viral treatments provided herein to simultaneously clean and disinfect the at least partially recellularized liver.

In some embodiments, the non-human animal organ is treated with one or more of a peracid, hydrogen peroxide, acetic acid, peracetic acid (PAA), saline, SDS, or sodium hydroxide (NaOH). In some embodiments, the non-human animal organ is treated with one or more chemicals selected from an acid (e.g., a peracid), hydrogen peroxide, a chemical compound comprising hydrogen peroxide, a chemical compound comprising a peracid, hydrogen peroxide covalently linked to an organic moiety, saline, and a sodium containing solution. In some embodiments, the non-human animal organ is treated with saline. In some embodiments, the non-human animal organ is treated with a solution comprising sodium, wherein the solution can comprise, for example, 0.1% NaCl, 0.2% NaCl, 0.5% NaCl, 0.7% NaCl, 0.8% NaCl, 0.9% NaCl, 1% NaCl, 1.5% NaCl, 2% NaCl 2.5% NaCl, 3% NaCl, 3.5% NaCl, 4% NaCl, 4.5% NaCl, 5% NaCl, 5.5% NaCl, 6% NaCl, 6.5% NaCl, 7% NaCl, 8% NaCl, 9% NaCl, 10% NaCl, 12% NaCl, 15% NaCl, 20% NaCl, 23% NaCl, or 25% NaCl. In some embodiments, the non-human animal is treated with a disinfection solution. In some embodiments, the disinfection solution can comprise saline and an acid. In some embodiments, a disinfection solution can comprise saline and peracid (e.g., peracetic acid). In some embodiments, a disinfection solution can comprise 0.9% NaCl and 600 ppm peracetic acid. In some embodiments, saline can be 1× saline, 2× saline, 5× saline, 7× saline, 10× saline, 12× saline, 15× saline. In some embodiments, a disinfection solution can comprise saline and a peracid (e.g., peracetic acid). In some embodiments, saline can be 1× saline. In some embodiments, an acid or peracid in a disinfection solution may range from about 25 parts per million (ppm) to about 4000 ppm. In some embodiments, an acid or peracid in a disinfection solution may range from about 500 ppm to about 700 ppm (500 ppm-700 ppm), 600 ppm-650 ppm, 250 ppm-700 ppm, 250 ppm-800 ppm, 550 ppm-1000 ppm, 600 ppm-700 ppm, 550 ppm-2000 ppm, 550 ppm-3000 ppm, 550 ppm-4000 ppm, 1000 ppm-2000 ppm, 2000 ppm-3000 ppm, or 3000 ppm-4000 ppm. In some embodiments, an acid or peracid can be about, at least about, or at most about 10 ppm, 25 ppm, 50 ppm, 75 ppm, 90 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 900 ppm, 1000 ppm, 1200 ppm, 1400 ppm, 1500 ppm, 1700 ppm, 2000 ppm, 2200 ppm, 2500 ppm, 2750 ppm, 3000 ppm, 3200 ppm, 3500 ppm, 3750 ppm, or 4000 ppm. In some embodiments, an acid or peracid (e.g., peracetic acid) can be about 600 ppm. In some embodiments, an acid or peracid (e.g., peracetic acid) can be about 50 ppm. In some embodiments, a disinfection solution may have a pH of about 4 to about 10 (e.g., pH of 6-7, 5-8, 5-10, 6-8, and 5.5-9). In some embodiments, a disinfecting solution has a pH of between about 5.00 to about 7.50, between about 6.00 to about 8.00, between about 6.10 to about 7.00, between about 4.50 to about 9.00, or between about 6.00 to about 6.50. In some embodiments, a disinfecting solution has a pH of about, at least about, or at most about 4.00, 4.50, 5.00, 5.50, 5.80, 5.90, 6.00, 6.05, 6.10, 6.11, 6.12, 6.13, 6.14, 6.15, 6.16, 6.17, 6.18, 6.19, 6.20, 6.30, 6.40, 6.41, 6.42, 6.43, 6.44, 6.45, 6.50, 6.60, 6.90, 7.00, 7.50, 8.00, 8.50, 9.0, 9.5, 10, 10.5, 11, 11.5, 12, 12.5, 13, 13.5, or 14. In some embodiments, disinfection of an isolated organ, a tissue, or a portion thereof may range from a few minutes, to days, to weeks, to months, or longer. In some embodiments, disinfection can be performed for about, at least about, or at most about 5 minutes, 10 min., 20 min., 30 min., 45 min., 1 hr, 1.5 hrs., 2 hrs., 2.5 hrs., 3 hrs., 3.5 hrs., 4 hrs., 4.5 hrs., 5 hrs., 5.5 hrs., 6 hrs., 6.5 hrs., 7 hrs., 7.5 hrs., 8 hrs., 8.5 hrs., 9 hrs., 9.5 hrs., 10 hrs., 12 hrs., 15 hrs., 18 hrs., 20 hrs., 22 hrs., 24 hrs., 27 hrs., 30 hrs., 34 hrs., 40 hrs., 44 hrs., 48 hrs., or longer. In some embodiments, disinfection can be performed for about, at least about, or at most about a day, two days, three days, four days, five days, six days, seven days, fourteen days, thirty days, or longer. In some embodiments, disinfection can be performed for about, at least about, or at most about a week, two weeks, three weeks, four weeks, five weeks, seven weeks, eight weeks, a month, two months, three months, four months, five months, six months, a year, or longer. In some embodiments, the non-human animal organ is treated with a peroxy acid or a hydrogen peroxide. In some embodiments, the peroxy acid comprises peroxyacetic acid, peracetic acid, peroxycarboxylic acid, derivatives, or combinations thereof.

### Organ Decellularization

Provided herein are at least partially recellularized organs (e.g. livers) or portions thereof, prepared from a decellularized extracellular matrix, wherein the decellularized extracellular matrix is from a non-human animal. Decellularization can be performed using methods described in U.S. Pat. Nos. 8,470,520, 11,278,643, U.S. application Ser. No. 17/781,843 and U.S. application Ser. No. 17/111,577, which are incorporated by reference herein in its entirety. In some embodiments, the decellularization comprises perfusion decellularization.

The initial step in decellularizing an organ or tissue, such as a liver, is to cannulate the organ or tissue, if possible. The vessels, ducts, and/or cavities of an organ or tissue can be cannulated using methods and materials known in the art. The next step in decellularizing an organ or tissue is to perfuse the cannulated organ or tissue with a cellular disruption medium. Perfusion through an organ can be multi-directional (e.g., antegrade and retrograde). Langendorff perfusion of a heart is routine in the art, as is physiological perfusion (also known as four chamber working mode perfusion). See, for example, Dehnert, The Isolated Perfused Warm-Blooded Heart According to Langendorff, In Methods in Experimental Physiology and Pharmacology: Biological Measurement Techniques V. Biomesstechnik-Verlag March GmbH, West Germany, 1988. Briefly, for Langendorff perfusion, the aorta is cannulated and attached to a reservoir containing cellular disruption medium. A cellular disruption medium can be delivered in a retrograde direction down the aorta either at a constant flow rate delivered, for example, by an infusion or roller pump or by a constant hydrostatic pressure. In both instances, the aortic valves are forced shut and the perfusion fluid is directed into the coronary ostia (thereby perfusing the entire ventricular mass of the heart), which then drains into the right atrium via the coronary sinus. For working mode perfusion, a second cannula is connected to the left atrium and perfusion can be changed from retrograde to antegrade.

Methods are known in the art for perfusing other organ or tissues. By way of example, the following references describe the perfusion of lung, liver, kidney, brain, and limbs. Van Putte et al., 2002, Ann. Thorac. Surg., 74(3):893-8; den Butter et al., 1995, Transpl. Int., 8:466-71; Firth et al., 1989, Clin. Sci. (Lond.), 77(6):657-61; Mazzetti et al., 2004, Brain Res., 999(1):81-90; Wagner et al., 2003, J. Artif. Organs, 6(3):183-91.

In some embodiments, one or more cellular disruption media can be used to decellularize an organ or tissue. A cellular disruption medium generally includes at least one detergent such as SDS, PEG, or Triton X. A cellular disruption medium can include water such that the medium is osmotically incompatible with the cells. Alternatively, a cellular disruption medium can include a buffer (e.g., PBS) for osmotic compatibility with the cells. Cellular disruption media also can include enzymes such as, without limitation, one or more collagenases, one or more dispases, one or more DNases, or a protease such as trypsin. In some instances, cellular disruption media also or alternatively can include inhibitors of one or more enzymes (e.g., protease inhibitors, nuclease inhibitors, and/or collegenase inhibitors).

In certain embodiments, a cannulated organ or tissue can be perfused sequentially with two different cellular disruption media. For example, the first cellular disruption medium can include an anionic detergent such as SDS and the second cellular disruption medium can include an ionic detergent such as Triton X. Following perfusion with at least one cellular disruption medium, a cannulated organ or tissue can be perfused, for example, with wash solutions and/or solutions containing one or more enzymes such as those provided herein. Alternating the direction of perfusion (e.g., antegrade and retrograde) can help to effectively decellularize the entire organ or tissue. Decellularization as provided herein essentially decellularizes the organ from the inside out, resulting in very little damage to the ECM. An organ or tissue can be decellularized at a suitable temperature between 4 and 40° C. Depending upon the size and weight of an organ or tissue and the particular detergent(s) and concentration of detergent(s) in the cellular disruption medium, an organ or tissue generally is perfused from about 0.1 to about 12 hours per gram of solid organ or tissue with cellular disruption medium. Including washes, an organ may be perfused for up to about 12 to about 72 hours per gram of tissue. Perfusion generally is adjusted to physiologic conditions including pulsatile flow, rate and pressure.

As provided herein, a decellularized organ or tissue consists essentially of the extracellular matrix (ECM) component of all or most regions of the organ or tissue, including ECM components of the vascular tree. ECM components can include any or all of the following: fibronectin, fibrillin, laminin, elastin, members of the collagen family (e.g., collagen I, III, and IV), glycosaminoglycans, ground substance, reticular fibers and thrombospondin, which can remain organized as defined structures such as the basal lamina. Successful decellularization is defined as the absence of detectable myofilaments, endothelial cells, smooth muscle cells, and nuclei in histologic sections using standard histological staining procedures. Preferably, but not necessarily, residual cell debris also has been removed from the decellularized organ or tissue.

In some embodiments, to effectively recellularize and generate an organ or tissue, it is important that the morphology and the architecture of the ECM be maintained (i.e., remain substantially intact) during and following the process of decellularization. “Morphology” as used herein can refer to the overall shape of the organ or tissue or of the ECM, while “architecture” as used herein can refer to the exterior surface, the interior surface, and the ECM therebetween.

The morphology and architecture of the ECM can be examined visually and/or histologically. For example, the basal lamina on the exterior surface of a solid organ or within the vasculature of an organ or tissue should not be removed or significantly damaged due to decellularization. In addition, the fibrils of the ECM should be similar to or significantly unchanged from that of an organ or tissue that has not been decellularized.

One or more compounds can be applied in or on a decellularized organ or tissue to, for example, preserve the decellularized organ, or to prepare the decellularized organ or tissue for recellularization and/or to assist or stimulate cells during the recellularization process. Such compounds include, but are not limited to, one or more growth factors (e.g., VEGF, DKK-1, FGF, bFGF, PDGF, HGF, BMP-1, BMP-4, SDF-1, IGF, and HGF), immune modulating agents (e.g., cytokines, glucocorticoids, IL2R antagonist, leucotriene antagonists, including but not limited to antibody therapy, use of stem cells to modulate the immune response, bone marrow transplant, etc.), and/or factors that modify the coagulation cascade (e.g., aspirin, heparin-binding proteins, and heparin). In addition, a decellularized organ or tissue can be further treated with, for example, irradiation (e.g., UV, gamma) to reduce or eliminate the presence of any type of microorganism remaining on or in a decellularized organ or tissue.

In some aspects, perfusion decellularization of an ECM from an organ or tissue can retain a native microstructure, such as an intact vascular and/or microvascular system, as compared to other decellularization techniques such as immersion based decellularization. For example, perfusion decellularized ECM from organs or tissues can preserve the collagen content and other binding and signaling factors and vasculature structure, thus providing for a niche environment with native cues for functional differentiation or maintenance of cellular function of introduced cells. In one embodiment, perfusion decellularized ECM from organs or tissues can be perfused with cells and/or media using the vasculature of the perfusion decellularized ECM under appropriate conditions, including appropriate pressure and flow to mimic the conditions normally found in the organism. The normal pressures of human sized organs can be between about 40 mm Hg to about 200 mm Hg with the resulting flow rate dependent upon the incoming perfusion vessel diameter. For a normal human heart the resulting perfusion flow is about 20 mL/min/100 g to about 200 mL/min/100 g. Using such a system, the seeded cells can achieve a greater seeding concentration of about 5× up to about 1000× greater than achieved under 2D cell culture conditions and, unlike a 2D culture system, the ECM environment allows for the further functional differentiation of cells, e.g., differentiation of progenitor cells into cells that demonstrate organ- or tissue-specific phenotypes having sustained function.

In some aspects, perfusion decellularization comprises cannulating an organ or portion thereof. In some aspects, at least one cannulation is introduced to an organ or portion thereof. In some aspects, at least two cannulations are introduced to an organ or portion thereof. In some aspects, from about 1, 2, 3, 4, 5, 6, 7, 8, 9, or up to 10 cannulations are introduced to an organ or portion thereof. In some cases, a cannula can be a part of a cannulation system. A cannulation system can comprise a size-appropriate hollow tubing for introducing into a vessel, duct, cavity, or any combination thereof of an organ or tissue. Typically, at least one vessel, duct, and/or cavity is cannulated in an organ. A perfusion apparatus or cannulation system can include a holding container for solutions (e.g., a cellular disruption medium) and a mechanism for moving the liquid through the organ (e.g., a pump, air pressure, gravity) via the one or more cannulae. The sterility of an organ or tissue during decellularization and/or recellularization can be maintained using a variety of techniques known in the art such as controlling and filtering the air flow and/or perfusing with, for example, antibiotics, anti-fungals or other anti-microbials to prevent the growth of unwanted microorganisms. In some aspects, a system as provided herein can possess the ability to monitor certain perfusion characteristics (e.g., pressure, volume, flow pattern, temperature, gases, pH), mechanical forces (e.g., ventricular wall motion and stress), and electrical stimulation (e.g., pacing). In some aspects, a vascular bed can change over the course of decellularization and recellularization (e.g., vascular resistance, volume), a pressure-regulated perfusion apparatus or cannulation system can be advantageous to avoid or reduce fluctuations. The effectiveness of perfusion can be evaluated in the effluent and in tissue sections. Perfusion volume, flow pattern, temperature, partial O₂ and CO₂ pressures and pH can be monitored using standard methods. In some aspects, sensors can be used to monitor the system (e.g., bioreactor) and/or the organ or tissue. Sonomicromentry, micromanometry, and/or conductance measurements can be used to acquire pressure-volume or preload recruitable stroke work information relative to myocardial wall motion and performance. For example, sensors can be used to monitor the pressure of a liquid moving through a cannulated organ or tissue; the ambient temperature in the system and/or the temperature of the organ or tissue; the pH and/or the rate of flow of a liquid moving through the cannulated organ or tissue; and/or the biological activity of a recellularizing organ or tissue. In addition to having sensors for monitoring such features, a system for decellularizing and/or recellularizing an organ or tissue also can include means for maintaining or adjusting such features. Means for maintaining or adjusting such features can include components such as a thermometer, a thermostat, electrodes, pressure sensors, overflow valves, valves for changing the rate of flow of a liquid, valves for opening and closing fluid connections to solutions used for changing the pH of a solution, a balloon, an external pacemaker, and/or a compliance chamber. To help ensure stable conditions (e.g., temperature), the chambers, reservoirs, and tubings can be water-jacketed.

In some aspects, a method of decellularization comprises providing an organ or portion thereof, cannulating the organ or portion thereof, and perfusing the cannulated organ or portion thereof with a solution or medium via the cannulation. In some aspects, the cannulation occurs at a cavity, vessel, duct, or combination thereof. In some aspects, from about 1 to 3, from about 1 to 5, from about 2 to 3, from about 2 to 5, from about 1 to 8 solutions can be utilized for organ perfusion. In some aspects, a solution is perfused at least two times. In some aspects, a solution is perfused at least 3, 4, 5, 6, 7, 8, 9, or up to 10 times through the organ or portion thereof. Various solutions and mediums can be employed during recellularization. In some aspects, a solution can be selected from the group comprising: cellular disruption solutions, washing solutions, disinfecting solutions, or combinations thereof.

In some aspects, a cellular disruption solutions is a solutions that can comprise at least one detergent, Table 1. A detergent can be an amphipathic molecule, that can contain both a nonpolar “tail” having aliphatic or aromatic character and a polar “head”. Ionic character of the polar head group can form the basis for broad classification of detergents; they may be ionic (charged, either anionic or cationic), nonionic (uncharged), or zwitterionic (having both positively and negatively charged groups but with a net charge of zero). In some aspects, detergents can be denaturing or non-denaturing with respect to protein structure. Denaturing detergents can be anionic such as sodium dodecyl sulfate (SDS) or cationic such as ethyl trimethyl ammonium bromide (ETMAB). These detergents totally disrupt membranes and denature proteins by breaking protein-protein interactions. Non-denaturing detergents can be divided into nonionic detergents such as Triton X-100, NP40, Tween, bile salts such as cholate, and zwitterionic detergents such as CHAPS.

TABLE 1
Detergents that can be utilized in cellular disruption solutions.

| | | Agg.# | | | | |
|---|---|---|---|---|---|---|
| | | (number of | | | | |
| | | molecules | MW | CMC | Cloud | |
| | | per | mono | mM | point | |
| Detergent | Type | micelle) | (micelle) | (% w/v) | ° C. | Dialyzable |
| Triton X-100 | Nonionic | 140 | 647 (90K) | 0.24 (0.0155) | 64 | No |
| Triton X-114 | Nonionic | — | 537 (—) | 0.21 (0.0113) | 23 | No |
| NP-40 | Nonionic | 149 | 617 (90K) | 0.29 (0.0179) | 80 | No |
| Brij-35 | Nonionic | 40 | 1225 (49K) | 0.09 (0.0110) | >100 | No |
| Brij-58 | Nonionic | 70 | 1120 (82K) | 0.08 (0.0086) | >100 | No |
| Tween 20 | Nonionic | — | 1228 (—) | 0.06 (0.0074) | 95 | No |
| Tween 80 | Nonionic | 60 | 1310 (76K) | 0.01 (0.0016) | — | No |
| Octyl glucoside | Nonionic | 27 | 292 (8K) | 23-24 (~0.70) | >100 | Yes |
| Octyl | Nonionic | — | 308 (—) | 9 (0.2772) | >100 | Yes |
| thioglucoside | | | | | | |
| SDS | Anionic | 62 | 288 (18K) | 6-8 (0.17-0.23) | >100 | No |
| CHAPS | Zwitterionic | 10 | 615 (6K) | 8-10 (0.5-0.6) | >100 | Yes |
| CHAPSO | Zwitterionic | 11 | 631 (7K) | 8-10 (~0.505) | 90 | Yes |

In some aspects, a washing solution may be utilized during decellularization. A washing solution may be utilized to remove residual solutions such as cellular disruption solutions from an organ or portion thereof as well as residual cellular components, enzymes, or combinations thereof. Suitable washing solutions may comprise water, filtered water, Phosphate buffered saline (PBS), and combinations thereof. PBS can maintain a constant pH and the osmolarity of cells. The pH of most biological materials falls from about 7 to about 7.6. Any concentration of PBS may be utilized as a washing solutions, PBS at 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 35%, 40%, 45%, 50%, 55%, 60%, 65%, 70%, 75%, 80%, 85%, 90%, 95%, or up to about 100%. In some aspects, a washing solution may be supplemented with agents. An agent can be an antibiotic, DNaseI, a disinfectant, and the like.

In some aspects, a disinfecting solution may be utilized during decellularization. A disinfecting solution may comprise any number of agents such as antibiotics, disinfectants, or combinations thereof. In some aspects, an antibiotic that can be used in a decellularization solution can be selected from the group comprising: actinomycin, ampicillin, carbenicillin, cefotaxime, fosmidomycin, gentamicin, kanamycin, neomycin, amphotericin, penicillin, polymyxin, streptomycin, broad selection antibiotic, and combinations thereof. Any concentration of antibiotic may be introduced in a disinfecting solution. Suitable concentrations of antibiotics can be: 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 35%, 40%, 45%, 50%, or up to about 60%. Suitable concentrations of antibiotics can be: 0.5 U/ml, 1 U/ml, 5 U/ml, 10 U/ml, 20 U/ml, 30 U/ml, 40 U/ml, 50 U/ml, 60 U/ml, 70 U/ml, 80 U/ml, 90 U/ml, 100 U/ml, 110 U/ml, 120 U/ml, 130 U/ml, 140 U/ml, 150 U/ml, 160 U/ml, 170 U/ml, 180 U/ml, 190 U/ml, 200 U/ml, 300 U/ml, 400 U/ml, 500 U/ml, 600 U/ml, 700 U/ml, 800 U/ml, 900 U/ml, 1000 U/ml, and up to about 1500 U/ml. Suitable concentrations of antibiotics can be: 0.5 μg/ml, 1 μg/ml, 1.5 μg/ml, 2 μg/ml, 2.5 μg/ml, 3 μg/ml, 3.5 μg/ml, 4 μg/ml, 4.5 μg/ml, 5 μg/ml, 5.5 μg/ml, 6 μg/ml, 6.5 μg/ml, 7 μg/ml, 7.5 μg/ml, 8 μg/ml, 8.5 μg/ml, 9 μg/ml, 9.5 μg/ml, 10 μg/ml, 15 μg/ml, 20 μg/ml, 25 μg/ml, 30 μg/ml, 35 μg/ml, 40 μg/ml, 45 μg/ml, 50 μg/ml, or up to about 60 μg/ml. In some aspects, an antibiotic may be 1% benzalkonium chloride, 100 U/ml penicillin-G, 100 U/ml streptomycin, and 0.25 μg/ml Amphotericin B.

Generally, moderate concentrations of mild (i.e., nonionic) detergents can compromise the integrity of cell membranes, thereby facilitating lysis of cells and extraction of soluble protein, often in native form. Using certain buffer conditions, various detergents effectively penetrate between the membrane bilayers at concentrations sufficient to form mixed micelles with isolated phospholipids and membrane proteins. In some aspects, denaturing detergents such as SDS can bind to both membrane (hydrophobic) and non-membrane (water-soluble, hydrophilic) proteins at concentrations below the CMC (i.e., as monomers). The reaction is equilibrium driven until saturated. Therefore, the free concentration of monomers determines the detergent concentration. SDS binding is cooperative (i.e., the binding of one molecule of SDS increases the probability that another molecule of SDS will bind to that protein) and alters most proteins into rigid rods whose length is proportional to molecular weight. In some aspects, non-denaturing detergents such as Triton X-100 have rigid and bulky nonpolar heads that do not penetrate into water-soluble proteins; consequently, they generally do not disrupt native interactions and structures of water-soluble proteins and do not have cooperative binding properties. The main effect of non-denaturing detergents is to associate with hydrophobic parts of membrane proteins, thereby conferring miscibility to them.

In some aspects, a system for generating an organ or portion thereof or tissue may be controlled by a computer-readable storage medium in combination with a programmable processor (e.g., a computer-readable storage medium as used herein has instructions stored thereon for causing a programmable processor to perform particular steps). For example, such a storage medium, in combination with a programmable processor, may receive and process information from one or more of the sensors. Such a storage medium in conjunction with a programmable processor also can transmit information and instructions back to the bioreactor and/or the organ or tissue. In some aspects, an organ or tissue undergoing recellularization may be monitored for biological activity. Biological activity can be that of the organ or portion thereof or tissue itself such as for cardiac tissue, electrical activity, mechanical activity, mechanical pressure, contractility, and/or wall stress of the organ or tissue. In addition, the biological activity of cells attached or engrafted on to the organ or portion thereof or tissue may be monitored, for example, for ion transport/exchange activity, cell division, and/or cell viability. In some aspects, it may be useful to simulate an active load on an organ or portion thereof during recellularization. In some aspects, a computer-readable storage medium of the invention, in combination with a programmable processor, may be used to coordinate the components necessary to monitor and maintain an active load on an organ or tissue. In some cases, the weight of an organ or portion thereof or tissue may be entered into a computer-readable storage medium as provided herein, which, in combination with a programmable processor, can calculate exposure times and perfusion pressures for that particular organ or tissue. Such a storage medium may record preload and afterload (the pressure before and after perfusion, respectively) and the rate of flow. In this embodiment, for example, a computer-readable storage medium in combination with a programmable processor can adjust the perfusion pressure, the direction of perfusion, and/or the type of perfusion solution via one or more pumps and/or valve controls.

In some aspects, perfusion decellularization of an organ or portion thereof can be from about 5%, 10%, 20%, 30%, 40%, 50%, 60%, 70%, 80%, 90%, 95%, or up to about 100% more effective as compared to a non-perfusion based decellularization system. Decellularization of an organ or portion thereof can be determined using various means. In some aspects, decellularization can be determined by histological examination. Histological examination can demonstrate the lack or reduction of cellular material, nuclei, and combinations thereof within a decellularized organ or portion thereof with preservation of the overall structure such as lobules and central veins. In some aspects, decellularization may be determined by immunohistochemical staining. Immunohistochemical staining can demonstrate paucity of cellular factors such as galactosyl-alpha (1,3) galactose (alpha-Gal) following perfusion decellularization. In some aspects, decellularization can be determined using DNA quantification. DNA quantification can comprise assays such as Picogreen. DNA quantification assays can determine an amount of a reduction of DNA in an organ or portion thereof.

A perfusion-based decellularized organ or portion thereof preserves a native scaffold containing the appropriate microenvironment required for the introduction of organ-specific cells, along with an intact vascular network to reconnect to a subject's blood supply and an outer capsule capable of maintaining physiologic pressures. These components are critical for the later use of perfusion recellularization, which also uses perfusion to repopulate vascular and organ-specific regenerative cells onto the organ, where they migrate to the appropriate microenvironment (via the relevant signaling protein markers that remain within the perfusion decellularized scaffold) as the organs are grown and matured in bioreactors under normal physiologic conditions. The resulting organs then can be transplanted utilizing the same techniques as current organ transplantation. Scaffolds created by perfusion decellularization are capable of receiving and incorporating a variety of cells on the organ scaffold utilized.

### Immersion Decellularization

In some aspects, immersion-based decellularization of an organ or portion thereof can be performed. In some aspects, whole organs or portions thereof can be decellularized by removing the entire cellular and tissue content from the organ. In some aspects, decellularization can comprise a series of sequential extractions. In some aspects, a first step can involve removal of cellular debris and solubilization of a cell membrane. This can be followed by solubilization of the nuclear cytoplasmic components and the nuclear components. In some aspects, an organ can be decellularized by removing the cell membrane and cellular debris surrounding the organ using gentle mechanical disruption methods. The gentle mechanical disruption methods can disrupt the cellular membrane. However, the process of decellularization should avoid damage or disturbance of the biostructure's complex infra-structure. Gentle mechanical disruption methods can include scraping the surface of the organ, agitating the organ, or stirring the organ in a suitable volume of fluid, e.g., distilled water. In some aspects, the gentle mechanical disruption method can include magnetically stirring (e.g., using a magnetic stir bar and a magnetic plate) the organ or portion thereof in a suitable volume of distilled water until the cell membrane is disrupted and the cellular debris has been removed from the organ or portion thereof. After the cell membrane has been removed, the nuclear and cytoplasmic components of the biostructure are removed. This can be performed by solubilizing the cellular and nuclear components without disrupting the infra-structure. To solubilize the nuclear components, non-ionic detergents or surfactants may be used. Examples of nonionic detergents or surfactants include, but are not limited to, the Triton series, available from Rohm and Haas of Philadelphia, Pa., which includes Triton X-100, Triton N-101, Triton X-114, Triton X-405, Triton X-705, and Triton DF-16, available commercially from many vendors; the Tween series, such as monolaurate (Tween 20), monopalmitate (Tween 40), monooleate (Tween 80), and polyoxethylene-23-lauryl ether (Brij. 35), polyoxyethylene ether W-1 (Polyox), and the like, sodium cholate, deoxycholates, CHAPS, saponin, n-Decyl β-D-glucopuranoside, n-heptyl β-D glucopyranoside, n-Octylα-D-glucopyranoside and Nonidet P-40.

### Physical Treatments

In some cases, physical treatment of an organ or portion thereof can be done to achieve decellularization. Physical treatment can be used to lyse, kill, and remove cells from an ECM or portion thereof. Physical treatment can utilize temperature, force, pressure, and electrical disruption. In some cases, temperature methods can be used in a rapid freeze-thaw mechanism. For example, by freezing a tissue, microscopic ice crystals can form around the plasma membrane and the cell can be lysed. After lysing the cells, the tissue can be further exposed to liquidized chemicals that can degrade and wash out any residual or undesirable components. In some cases, temperature methods can conserve the physical structure of the ECM scaffold. An organ or portion thereof, and a tissue can be decellularized at a suitable temperature. A suitable temperature can be from about 4° C., 8° C., 10° C., 12° C., 14° C., 16° C., 18° C., 20° C., 22° C., 24° C., 26° C., 28° C., 30° C., 32° C., 34° C., 36° C., 38° C., 40° C., 45° C., 50° C., 55° C., 60° C., or up to about 70° C. A physical treatment can also include the use of pressure. Pressure decellularization can involve the controlled use of hydrostatic pressure applied to a tissue, organ, or portion thereof. Pressure decellularization can be performed at high temperatures in some cases to avoid unmonitored ice crystal formation. In some cases, Electrical disruption of an organ or portion thereof can be performed. Electrical disruption can be done to lyse cells housed in a tissue or organ. By exposing a tissue, organ, or portion thereof to electrical pulses, micropores can be formed at the plasma membrane. The cells can die after their homeostatic electrical balance is ruined through the applied stimulus. This electrical process is documented as Non-thermal irreversible electroporation (NTIRE).

### Chemical and Enzymatic Treatments

In some cases, chemical treatment of an organ or portion thereof can be performed to achieve decellularization. Chemicals and/or salts thereof for use in a chemical treatment can be selected for decellularization depending on the thickness, extracellular matrix composition, and intended use of the tissue or organ. For example, enzymes would not be used on a collagenous tissue because they disrupt the connective tissue fibers. However, when collagen is not present in a high concentration or needed in the tissue, enzymes can be a viable option for decellularization. The chemicals and/or salts thereof can be used to kill and remove cells can be but are not limited to acids, alkaline treatments, ionic detergents, non-ionic detergents, and zwitterionic detergents. In some cases, one or more chemicals can comprise a cellular disruption media. A cellular disruption medium can comprise at least one detergent such as Sodium dodecyl sulfate (SDS), polyethyleneglycol (PEG), or Triton X. Detergents can act effectively to lyse the cell membrane and expose the contents to further degradation. For example, after SDS lyses a cellular membrane, endonucleases and/or exonucleases can degrade the genetic contents, while other components of the cell can be solubilized and washed out of the matrix. In some cases, a detergent can be mixed with an alkaline and/or acid treatments due to their ability to degrade nucleic acids and solubilize cytoplasmic inclusions.

One or more cellular disruption media can be used to decellularize an organ or tissue. A cellular disruption medium can comprise at least one detergent such as SDS, PEG, or Triton X. A cellular disruption medium can comprise water such that the medium is osmotically incompatible with the cells. Alternatively, a cellular disruption medium can comprise a buffer (e.g., PBS) for osmotic compatibility with the cells. Cellular disruption media also can include enzymes such as, without limitation, one or more collagenases, one or more dispases, one or more DNases, one or more proteases, and any combination thereof. In some instances, cellular disruption media also or alternatively can include inhibitors of one or more enzymes (e.g., protease inhibitors, nuclease inhibitors, and/or collegenase inhibitors). A cellular disruption medium can include water such that the medium is osmotically incompatible with the cells. Alternatively, a cellular disruption medium can include a buffer (e.g., PBS) for osmotic compatibility with the cells. Cellular disruption media also can include enzymes such as, without limitation, one or more collagenases, one or more dispases, one or more DNases, or a protease such as trypsin. In some instances, cellular disruption media also or alternatively can include inhibitors of one or more enzymes (e.g., protease inhibitors, nuclease inhibitors, and/or collegenase inhibitors). In some cases, a non-ionic detergent such as Triton X-100 can be utilized. Triton X-100 can disrupt the interactions between lipids and between lipids and proteins. In some cases, Triton X-100 may not disrupt protein-protein interactions, which can be beneficial to keeping the ECM intact. In some cases, EDTA can be utilized. EDTA can be a chelating agent that binds calcium, which can be a component for proteins to interact with one another. By making calcium unavailable, EDTA can prevent the integral proteins between cells from binding to one another. EDTA can be used with trypsin, an enzyme that acts as a protease to cleave the already existing bonds between integral proteins of neighboring cells within a tissue.

A detergent can be administered from about 10 min, 30 min, 60 min, 1 hr., 2 hrs., 3 hrs., 4 hrs., 5 hrs., 6 hrs., 7 hrs., 8 hrs., 9 hrs., 10 hrs., 11 hrs., 12 hrs., 13 hrs., 14 hrs., 15 hrs., 16 hrs., 17 hrs., 18 hrs., 19 hrs., 20 hrs., 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs., 44 hrs., 45 hrs., 46 hrs., 47 hrs., 48 hrs., 49 hrs., 50 hrs., 51 hrs., 52 hrs., 53 hrs., 54 hrs., 55 hrs., 56 hrs., 57 hrs., 58 hrs., 59 hrs., 60 hrs., 70 hrs., 80 hrs., 90 hrs., or up to about 100 hrs.

Depending upon the size and/or weight of an organ or portion thereof a chemical treatment such as a detergent can be contacted with the organ or portion thereof from about 2 hours, 3 hours, 4 hours, 5 hours, 6 hours, 7 hours, 8 hours, 9 hours, 10 hours, 11 hours, 12 hours, 13 hours, 14 hours, 15 hours, to about 20 hours per gram of solid organ or tissue with cellular disruption medium.

Including washes, an organ may be perfused for up to about 12 hrs., 13 hrs., 14 hrs., 15 hrs., 16 hrs., 17 hrs., 18 hrs., 19 hrs., 20 hrs., 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs., 44 hrs., 45 hrs., 46 hrs., 47 hrs., 48 hrs., 49 hrs., 50 hrs., 51 hrs., 52 hrs., 53 hrs., 54 hrs., 55 hrs., 56 hrs., 57 hrs., 58 hrs., 59 hrs., 60 hrs., 70 hrs., 80 hrs., 90 hrs., or up to about 100 hrs. In some cases, an organ or portion thereof can be perfused from about 12 hours to about 72 hours per gram of tissue. In some aspects, perfusion can be adjusted to physiologic conditions including pulsatile flow, rate, pressure, and any combination thereof.

In some cases, an organ, portion thereof, or tissue can be contacted sequentially with at least two different cellular disruption media. For example, the first cellular disruption medium can include an anionic detergent such as SDS and the second cellular disruption medium can include an ionic detergent such as Triton X. Following contacting, such as perfusion, with at least one cellular disruption medium, a cannulated organ or tissue can be perfused, for example, with wash solutions and/or solutions containing one or more enzymes such as those provided herein. In some cases, alternating the direction of perfusion (e.g., antegrade and retrograde) can help to effectively decellularize an organ, portion thereof, or tissue. Decellularization as provided herein can decellularize an organ or portion thereof from the inside out, resulting in very little damage to the ECM.

In some cases, a sequential method of decellularization can comprise contacting the organ or portion thereof with a cellular disruption media, such as an SDS detergent, followed by a washing step, followed by the addition of one or more chemicals, followed by contacting with a detergent, and ending with at least one wash step. A sequential method of decellularization can comprise at least 1, 2, 3, 4, 5, 6, 7, 8, 9, 10, 11, 12, 13, 14, or up to 15 contacting steps with any media or solution provided herein.

A buffer provided herein can be at a concentration from about 0.1%, 0.5%, 1%, 1.5%, 2%, 2.5%, 3%, 3.5%, 4%, 4.5%, 5%, 10%, 20%, 30%, 40%, 50%, 60%, 70%, 80%, 90%, 95%, or up to about 100%.

### Organ Recellularization

Decellularized organs and portions thereof provided herein can be recellularized. An organ or tissue can be generated by contacting a decellularized organ or tissue, for example, a decellularized non-human animal liver, as provided herein with a population of cells. In some embodiments, the decellularized extracellular matrix provided herein is contacted with a cell composition comprising one or more populations of cells or cell types. In some embodiments, the decellularized extracellular matrix provided herein is contacted with a first cell composition and a second cell composition. In some embodiments, the first cell composition comprises a population of endothelial cells. In some embodiments, the second cell composition comprises a population of liver cells. In some embodiments the cell composition provided herein is a mixture of different cell types. For example, a cell composition can comprise a population of liver cells and a population of endothelial cells.

Provided herein are at least partially recellularized livers that comprise a first cell composition comprising human endothelial cells and a second cell composition comprising human liver cells. In some embodiments, the decellularized extracellular matrix is contacted with the second cell composition when the first cell composition is characterized as having a glucose consumption rate of at least about 10 mg/hr, at least about 20 mg/hr, at least about 30 mg/hr, at least about 40 mg/hr, at least about 50 mg/hr, at least about 60 mg/hr, at least about 70 mg/hr, at least about 80 mg/hr, at least about 90 mg/hr, or at least about 100 mg/hr.

In some embodiments, a cell composition can comprises a population of regenerative cells. Regenerative cells as used herein are any cells used to recellularize a decellularized organ or tissue. Regenerative cells can be totipotent cells, pluripotent cells, or multipotent cells, and can be uncommitted or committed. Regenerative cells also can be single-lineage cells. In addition, regenerative cells can be undifferentiated cells, partially differentiated cells, or fully differentiated cells. Regenerative cells as used herein include embryonic stem cells (as defined by the National Institute of Health (NIH); see, for example, the Glossary at stemcells.nih.gov on the World Wide Web). Regenerative cells also include progenitor cells, precursor cells, and “adult”-derived stem cells including umbilical cord cells and fetal stem cells. Examples of regenerative cells that can be used to recellularize an organ or portion thereof provided herein can be, without limitation, embryonic stem cells, umbilical cord blood cells, tissue-derived stem or progenitor cells, bone marrow-derived stem or progenitor cells, blood-derived stem or progenitor cells, adipose tissue-derived stem or progenitor cells, mesenchymal stem cells (MSC), skeletal muscle-derived cells, induced pluripotent stem cells (iPSCs), genetically modified cells removing immunogenic factors including but not limited to HLA, or multipotent adult progenitor cells (MAPC). Additional regenerative cells that can be used include tissue-specific stem cells including cardiac stem cells (CSC), multipotent adult cardiac-derived stem cells, cardiac fibroblasts, cardiac microvasculature endothelial cells, or aortic endothelial cells. Bone marrow-derived stem cells such as bone marrow mononuclear cells (BM-MNC), endothelial or vascular stem or progenitor cells, and peripheral blood-derived stem cells such as endothelial progenitor cells (EPC) also can be used as regenerative cells. In some aspects, the number of regenerative cells that can be introduced into a decellularized organ or portion thereof in order to generate an organ or tissue can be dependent on both the organ (e.g., which organ, the size and weight of the organ) or tissue and the type and developmental stage of the regenerative cells. Different types of cells may have different tendencies as to the population density those cells will reach. Similarly, different organ or tissues may be recellularized at different densities. By way of example, a decellularized organ or tissue can be “seeded” with at least about 1,000 (e.g., at least 10,000, 100,000, 1,000,000, 10,000,000, or 100,000,000) regenerative cells; or can have from about 1,000 cells/mg tissue (wet weight, i.e., prior to decellularization) to about 10,000,000 cells/mg tissue (wet weight) attached thereto. In some aspects, regenerative cells can be introduced (“seeded”) into a decellularized organ or tissue by injection into one or more locations.

The methods of recellularizing a tissue or organ matrix as provided herein also include re-endothelialization of the tissue or organ matrix with endothelial cells or endothelial progenitor cells. In one embodiment, endothelial cells and endothelial progenitor cells are obtained by culturing embryonic stem cells (ESCs) or induced pluripotent stem cells (iPSCs) under appropriate conditions to direct the stem cells down an endothelial lineage. Endothelial progenitor cells are cells that have begun to differentiate into endothelial cells or have the potential to (e.g., multi-potent; e.g., lineage-restricted; e.g., cells that are destined to become endothelial cells) but are not considered fully differentiated endothelial cells. For example, endothelial cells typically express platelet endothelial cell-adhesion molecule-1 (PECAM1; aka CD31) and may also express one or more of the following markers: VEGFR-1 (aka Flt-1), VEGFR-2 (aka Flk-1), guanylate-binding protein-1 (GBP-1), thrombomodulin (aka CD141), VE-cadherin (aka CD144), von Willebrand factor (vWF), and intercellular adhesion molecule 2 (ICAM-2). Generally, endothelial progenitor cells also are able to take up acetylated LDL, and, further, may migrate toward VEGF and/or form tubes on a Matrigel.

ESCs or iPSCs can be further cultured under conditions that result in fully differentiated endothelial cells. Additionally or alternatively, endothelial cells can be obtained from any number of sources such as blood, skin, liver, heart, lung, retina, and any other tissue or organ that harbors endothelial cells. For example, representative endothelial cells include, without limitation, blood endothelial cells, bone marrow endothelial cells, circulating endothelial cells, human aorta endothelial cells, human brain microvascular endothelial cells, human dermal microvascular endothelial cells, human intestinal microvascular endothelial cells, human lung microvascular endothelial cells, human microvascular endothelial cells, hepatic sinusoidal endothelial cells, human saphenous vein endothelial cells, human umbilical vein endothelial cells, lymphatic endothelial cells, microvessel endothelial cells, microvascular endothelial cells, pulmonary artery endothelial cells, retinal capillary endothelial cells, retinal microvascular endothelial cells, vascular endothelial cells, umbilical cord blood endothelial cells, and combinations thereof. As those of skill in the art would understand, this is not intended to be an exhaustive list of endothelial cells.

Endothelial cells can be obtained, for example, from one of the many depositories of biological material around the world. See, for example, the American Type Culture Collection (ATCC.org on the World Wide Web) or the International Depositary Authority of Canada (IDAC; nml-lnm.gc.ca on the World Wide Web). Endothelial cells or endothelial progenitor cells also can be obtained from the individual that will be the recipient of the transplanted tissue or organ matrix. These cells would be considered to be autologous to the recipient. Additionally, under certain circumstances, the relationship between the tissue or organ matrix and the endothelial cells or endothelial progenitor cells can be allogeneic (i.e., different individuals from the same species); in other instances, the relationship between the tissue or organ matrix and the endothelial cells or endothelial progenitor cells can be xenogeneic (i.e., individuals from different species).

A composition that includes endothelial cells or endothelial progenitor cells typically is delivered to a tissue or organ matrix in a solution that is compatible with the cells (e.g., in a physiological composition) under physiological conditions (e.g., 37° C.) and under non-physiologic conditions (e.g. 4-35° C.).). A physiological composition, as referred to herein, can include, without limitation, buffers, nutrients (e.g., sugars, carbohydrates), enzymes, expansion and/or differentiation medium, cytokines, antibodies, repressors, growth factors, salt solutions, or serum-derived proteins. As used herein, a composition that “consists essentially of” endothelial cells or endothelial progenitor cells is a composition that is substantially free of cells other than endothelial cells or endothelial progenitor cells but may still include any of the components that might be found in a physiological composition (e.g., buffers, nutrients, etc.).

To optimize re-endothelialization, endothelial cells or endothelial progenitor cells generally are introduced into an organ or tissue matrix by perfusion. As with the pre-cellular perfusion, and as described in WO 2007/025233, perfusion occurs via the vasculature or vasculature-type structure of the organ or tissue matrix. Perfusion to re-endothelialize an organ or tissue matrix should be at a flow rate that is sufficient to circulate the physiological composition of cells through the vasculature. Perfusion with the endothelial cells or endothelial progenitor cells can be multi-directional (e.g., antegrade and retrograde) to even further optimize re-endothelialization. Perfusion of cells may be followed by a static hold time to enhance engraftment prior to reperfusion of the organ or tissue matrix.

In some aspects, at least one type of cell can be introduced into a decellularized organ or portion thereof. For example, a population of cells can be injected at multiple positions in a decellularized organ or tissue or different cell types can be injected into different portions of a decellularized organ or portion thereof. Alternatively, or in addition to injection, regenerative cells, a population of cells, or a cocktail of cells can be introduced by perfusion into a cannulated decellularized organ or portion thereof. For example, regenerative cells can be perfused into a decellularized organ using a perfusion medium, which can then be changed to an expansion and/or differentiation medium to induce growth and/or differentiation of the regenerative cells. During recellularization, an organ or tissue can be maintained under conditions in which at least some of the regenerative cells can proliferate, multiply, differentiate, and any combination thereof in the decellularized organ or portion thereof. In some aspects, those conditions can include, without limitation, the appropriate temperature, pressure, electrical activity, mechanical activity, force, the appropriate amounts of O₂ and/or CO₂, an appropriate amount of humidity, sterile or near-sterile conditions, and any combination thereof. During recellularization, the decellularized organ or tissue and the regenerative cells attached thereto can be maintained in a suitable environment. For example, the regenerative cells may require a nutritional supplement (e.g., nutrients and/or a carbon source such as glucose), exogenous hormones or growth factors, and/or a particular pH.

In some aspects, regenerative cells as provided herein can be allogeneic to a decellularized organ or portion thereof (e.g., a human decellularized organ or tissue seeded with human regenerative cells), or regenerative cells can be xenogeneic to a decellularized organ or portion thereof (e.g., a pig decellularized organ or tissue seeded with human regenerative cells). “Allogeneic” as used herein refers to cells obtained from the same species as that from which the organ or tissue originated (e.g., self (i.e., autologous) or related or unrelated individuals), while “xenogeneic” as used herein refers to cells obtained from a species different than that from which the organ or tissue originated.

In some embodiments, an endothelial cell can be perfused into a decellularized liver. Populations of endothelial cells may engraft onto the decellularized liver matrix as described above. In some embodiments, a recellularized organ may comprise a fenestrated endothelium that was absent from the decellularized liver, and or absent in the seeded or introduced cell population but may be present following engraftment and/or migration into the decellularized liver matrix.

Provided herein are at least partially recellularized livers that comprise human liver cells. In some embodiments, the human liver cells are primary human liver cells. In some embodiments, the human liver cells are derived from or differentiated from a human stem cell. In some embodiments, the human stem cell is an embryonic stem cell, an induced pluripotent stem cell, an adult stem cells. In some embodiments, the human liver cells are derived from or differentiated from a progenitor cell. In some embodiments, the human liver cells are in-vitro differentiated human liver cells.

In some embodiments, a decellularized extracellular matrix is coated with an additional extracellular matrix protein or solution. For example, the additional extracellular matrix protein or solution can include, but are not limited to: fibronectin, fibrillin, laminin, elastin, members of the collagen family (e.g., collagen I, III, and IV), and solubilized basement membrane matrix secreted by Engelbreth-Holm-Swarm mouse sarcoma cells (MATRIGEL®).

### Characterization of Recellularized Organs.

Provided herein are functional liver tissue and at least partially recellularized livers that clear ammonia, produce urea, and express both endothelial and hepatocyte surface markers.

The recellularized organs and compositions provided herein can be assayed for a set of cell surface markers and/or genes that are associated with the corresponding primary organ (e.g., a liver). The recellularized organs and compositions provided herein can be assayed relative to a population of cells that are not engrafted to a decellularized extracellular matrix.

The transformation or plasticity of an at least partially decellularized liver provided herein can be monitored by determining an expression level of certain genes or proteins. For example, genes in a parenchymal or sinusoidal niche of the recellularized organ can be used to evaluate the formation and structure of a recellularized organ. In some embodiments, a gene marker that can be used to determine sinusoidal marker expression which can be but not limited to VEGFR-3, D2-40, STAB2, CD31, RPL19, or LYVE-1. In some cases, who biopsies of the seeded liver graft can be taken and look for the expression of sinusoidal markers including but not limited to VEGFR-3, D2-40, STAB2, CD31, RPL19, or LYVE-1. In some cases, increased expression of sinusoidal genes and or the direct detection of fenestration measured following engraftment and/or migration and/or proliferation into the parenchymal space.

In some embodiments, the at least partially recellularized livers provided herein comprise one or more surface markers selected from the group consisting of: CD31, CD105, and asialoglycoprotein receptor 1 (ASGR1). In some embodiments, the at least partially recellularized livers provided herein comprise two or more surface markers selected from the group consisting of: CD31, CD105, and asialoglycoprotein receptor 1 (ASGR1). In some embodiments, the at least partially recellularized livers provided herein comprise a surface marker selected from the group consisting of: CD31, CD105, albumin, van Willebrand factor (vWF), Lymphatic Vessel Endothelial hyaluronan receptor 1 (LYVE-1), TEK receptor tyrosine kinase (Tie-2), and asialoglycoprotein receptor 1 (ASGR1). Methods of measuring gene expression include for example, polymerase chain reaction (PCR), microarrays, sequencing, Northern or Southern blotting techniques. Methods of measuring or tracking protein expression include, for example, Western blotting, immunosorbent assays (e.g., enzyme-linked immunosorbent assays or ELISA), flow cytometry, and microscopy techniques.

The engineered, recellularized livers provided herein have a functional urea cycle in which waste (ammonia) is removed from the blood by being metabolized to urea. Ammonia originates from protein catabolism whether that is secondary to a high-protein diet, deaminations, or during the period of prolonged starvation. Ammonia is also naturally produced by gut flora. In muscle and peripheral tissues, glutamate is the amino acid that accepts free ammonia, which results in the formation of glutamine. Glutamine is then exported from muscle and peripheral tissues and utilized by the liver. Glutaminase breaks down glutamine into glutamate and ammonia. Glutamate also yields additional urea via the enzyme glutamate dehydrogenase. From here, ammonia is initially incorporated into hepatocyte mitochondria and ultimately results in the formation of urea. Urea subsequently leaves the hepatocyte cytoplasm and is ultimately excreted in the urine.

Provided herein are at least partially recellularized livers characterized as having an increase in the level of ammonia clearance relative to a population of liver cells that are not engrafted onto a porcine extracellular matrix. In some embodiments, the at least partially recellularized livers are characterized as having ammonia clearance following cold storage. In some embodiments, the at least partially recellularized livers are in cold storage for 6 hours or more and have ammonia clearance. Methods of measuring ammonia clearance comprise an ammonia clearance assay described in the Examples.

Provided herein are at least partially recellularized livers characterized as having urea production. Further provided herein are at least partially recellularized livers characterized as having urea production that is increased relative to a population of liver cells that are not engrafted onto a porcine extracellular matrix. In some embodiments, the at least partially recellularized livers are characterized as having urea production following cold storage. In some embodiments, the at least partially recellularized livers are in cold storage for 6 hours or more and have urea production.

Provided herein are at least partially recellularized livers characterized as having an increase in the level of alpha-1-antitrypsin (A1AT) relative to a population of liver cells that are not engrafted onto a porcine extracellular matrix. A1AT is a protease inhibitor produced by the liver that protects tissues in the body from inflammation and improves lung elasticity. Loss of A1AT is associated can lead to a chronic uninhibited tissue breakdown.

Provided herein are at least partially recellularized livers characterized as having an increase in the level of fibrinogen relative to a population of liver cells that are not engrafted onto a porcine extracellular matrix. Fibrinogen is produced by the liver to modulate blood clotting (coagulation).

Provided herein are at least partially recellularized livers characterized as having a decrease in the level of microbial proteins or activity relative to an at least partially recellularized liver that has not been treated with an anti-viral treatment (e.g., an electron beam and PAA). The at least partially recellularized livers provided herein are able to maintained in culture for long periods of time (e.g., greater than 4 days) due to the improved overall health and safety of the recellularized organ.

Methods of measuring urea production, microbial proteins, microbial activity, fibrinogen, and A1AT comprise, for example, Western blot assays, colorimetric assays, chemical assays, liquid chromatography, or immunosorbent assays (e.g., ELISA). Additional methods are provided in the Examples further below.

Patency of a recellularized organ provided herein can be assessed over time. In some cases, patency can be assessed for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours. In some cases, patency can be assessed for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

In some embodiments, functionality of a recellularized organ provided herein is assessed by determining the consumption of certain metabolites (i.e. glucose, lactate, glutamine, glutamate and ammonia). Such consumption can be determined by perfusing in a continuous line of the metabolite and measuring a rate of consumption of the metabolite over time using, for example, a change in electrochemical potential. Methods and for detection of these metabolites are readily apparent to a skilled artisan, and sensors for determining these metabolites are readily available.

In some embodiments, the rate of consumption of a metabolite can be used to determine successful engraftment of endothelial cells onto a decellularized matrix. For example, a glucose consumption rate can be correlated to successful endothelialization in a recellularized liver. Furthermore, glucose consumption rate can be correlated to in vivo graft patency, thus enabling a glucose consumption rate to be used a surrogate for in vivo patency. Other metabolite consumption such as lactate, glutamine, glutamate and ammonia are expected to be predictive of in vivo patency.

### Uses of Organs and Portions Thereof.

Decellularized and recellularized organs or portions thereof provided herein can be used in a variety of applications. For example, organs or portions thereof can be implanted into a subject or used ex-vivo in a blood circuit that performs liver clearance of the blood from ammonia and other toxins.

### Ex-Vivo Blood Circuits

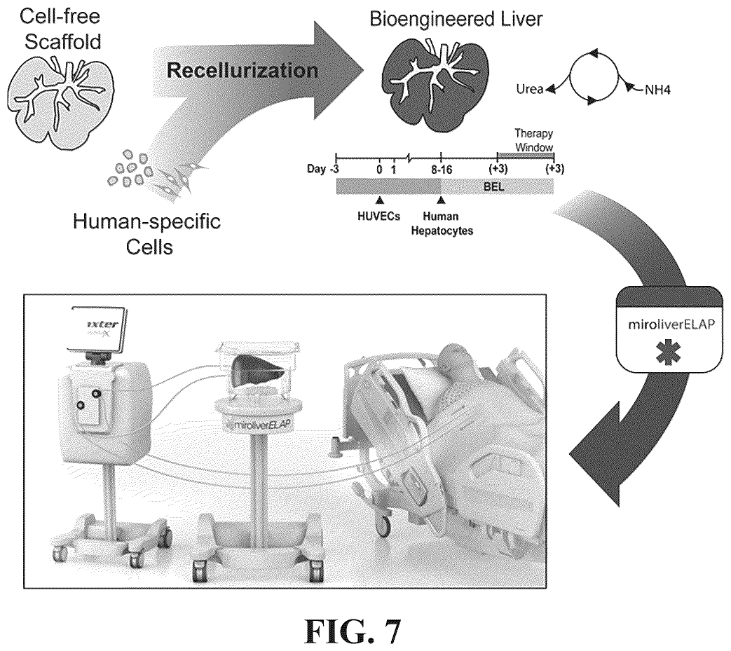

Provided herein are methods of treating a subject using an ex-vivo blood circuit. In some embodiments, the methods comprise producing a blood circuit, wherein the blood circuit comprises blood from the subject in fluid communication with an at least partially recellularized liver provided herein, wherein the at least partially recellularized liver filters blood from the subject and clears ammonia, thereby treating the liver disease in the subject. An ex-vivo blood perfusion circuit can be generated to filter the subject's blood and clear the blood of ammonia. An exemplary ex-vivo blood perfusion circuit is shown in FIG. 7. The ex-vivo blood circuit can be used, for example, while a subject is waiting for an organ transplantation, to assist with organ transplantation, or to prolong survival of a subject with a lethal disease.

In some embodiments, the ex-vivo circuit comprises a bioreactor. A bioreactor may be utilized as part of a system or ex-vivo blood circuit provided herein to supply an organ or portion thereof (e.g., an at least partially recellularized liver) with physical stimulation, electrical stimulation, chemical stimulation, or a combination of these. In some embodiments, a bioreactor can comprise means for increasing the level of oxygen in a culture media. In some embodiments, heightened oxygen levels can range from about 22% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, from about 95% to about 100%. In some embodiments, a level of increased oxygen can vary over culture time. n some embodiments, elevated levels of oxygen in a media in which cells are cultured can result in a decrease in glucose consumption, delayed metabolic switching to glucogenesis, decrease in lactate production, decrease in ammonia concentrations, increased ammonia clearance rates, stability of mature phenotype, proliferation, or any combination thereof. In some embodiments, elevated levels of oxygen in a media in which cells, such as hepatocytes, are cultured in can result in a decrease in glucose consumption, phenotypic stability as measured by metabolic activity, decreased or delayed glucogenesis, secretion of coagulation factors, decrease in lactate production, decrease in ammonia concentrations, increased ammonia clearance, or any combination thereof. In some embodiments, the presence of heightened oxygen levels in a media in which one or more cells, such as hepatocytes, are cultured can extend the ability to clear ammonia. In some embodiments, a heightened oxygen level can be produced by direct oxygenation, in-line oxygenation, gas permeable materials, or any combination thereof. In some cases, direct oxygenation can comprise using a membrane oxygenating chamber. In some embodiments, direct oxygenating can comprise using a bubbler. In some embodiments, in-line oxygenation can comprise use of in-line oxygenators. In some embodiments, a media may be pumped by a peristaltic pump. In some embodiments, a media may be passed through gas permeable material allowing gaseous exchange through the material. In some embodiments, gaseous exchange may comprise oxygen exchange, nitrogen exchange, carbon dioxide exchange, or any combination thereof. In some embodiments, an at least partly permeable tubing may comprise silicone tubing. In some embodiments, silicone tubing may allow oxygen exchange, creating heightened oxygen levels in a media. In some embodiments, oxygen levels in the media can be heightened by a direct injection of a mixture of oxygen and one or more other gases. In some embodiments, one or more other gases can comprise nitrogen, carbon dioxide, or a combination of the two. In some embodiments, oxygen levels in the media can be heightened by an injection of a gas comprising about 40% oxygen, about 45% oxygen, about 50% oxygen, about 55% oxygen, about 60% oxygen, about 65% oxygen, about 70% oxygen, about 75% oxygen, about 80% oxygen, about 85% oxygen, about 90% oxygen, about 95% oxygen, or about 100% oxygen. In some embodiments, oxygen levels in the media can be heightened by an injection of about 100% pure oxygen. In some embodiments, heightened oxygen levels in the media can be facilitated by oxygen carrying molecules to increase access to cells within an isolated organ or portion thereof. In some cases, cells may comprise seeded hepatocytes. In some cases, an isolated organ or portion thereof may comprise an extracellular matrix (ECM) graft. In some cases, a media can be hyperoxygenated prior to seeding cells into an isolated organ or portion thereof. In some cases, a media can be hyperoxygenated prior to seeding hepatocytes into an ECM graft. In some cases, oxygen levels can be adjusted based on metrics. In some cases, metrics can be evaluated or adjusted and can comprise media glucose levels, lactate levels, pCO2, pH, ammonia levels, pyruvate levels, other measurable parameters, and any combination thereof. In some embodiments, disclosed herein may be a system comprising any of the compositions disclosed herein. In some embodiments, a system may comprise at least one of a bioreactor, pump, housing, tubing, oxygen permeable tubing, incubator, motor, computer, storage medium, biological safety cabinet, incubator, or any combination thereof. In some embodiments, cells are stored in an incubator. In some embodiments, an incubator can regulate temperature, gaseous concentration, humidity, and any combination thereof.

### Organ Transplantation

In some aspects, a composition provided herein, such as a partially recellularized organ (e.g. liver), may be transplanted into a subject that has a disease. Relevant diseases that may require organ transplantation include but are not limited to: organ failure, cardiomyopathy, cirrhosis, chronic obstructive pulmonary disease, pulmonary edema, biliary atresia, emphysema and pulmonary hypertension, coronary heart disease, valvular heart disease, congenital heart disease, coronary artery disease, pancreatitis, cystic fibrosis, diabetes, hepatitis, hypertension, idiopathic pulmonary fibrosis, polycystic kidneys, short gut syndrome, injury, birth defects, genetic diseases, autoimmune disease, and any combination thereof. Implants, according to the invention, can be used to replace or augment existing tissue. For example, to treat a subject with a kidney disorder by replacing the dysfunctional kidney of the subject with an exogenous or engineered kidney. The subject can be monitored after implantation of the exogenous kidney, for amelioration of the kidney disorder. Any decellularized organ or potion thereof provided herein can be utilized for implantation into a subject.

In some aspects, a composition provided herein, such as a solid organ or portion thereof can have from about 1% to about 100% of its native function after decellularization. In some aspects, a composition provided herein, such as a solid organ or portion thereof can have from about 5%, 10%, 20%, 30%, 40%, 50%, 60%, 70%, 80%, 90%, 95%, or up to about 100% of its native function after decellularization.

In some aspects, particular organs or portions thereof may be suitable for transplantation when they function below that of their native counterpart. For example, a liver and a kidney may need approximately from about 20% of the total organ function to provide the needed organ function to save a person from liver failure or remove them from dialysis. In some aspects, a liver and kidney may need approximately from about 20-30%, 30-40%, 20-50%, 20-60%, 40-60% of the total organ function to be suitable for transplantation. In some aspects, an organ may function equal to a native counterpart. For example, a heart is more complicated, in that, it may need from about 80%, 85%, 90%, 95%, 96%, 97%, 98%, 99%, or up to about 100% function at the time of transplantation.

Provided herein are also compositions and methods of generating engineered organs or portions thereof comprising a population of cells. In some aspects, at least two populations of cells can be introduced into a decellularized organ or portion thereof. Organs that can be engineered include, but are not limited to, heart, kidney, liver, pancreas, spleen, urinary bladder, ureter, urethra, skeletal muscle, small and large bowel, esophagus, stomach, brain, spinal cord and bone.

In some cases, a recellularized liver can be transplanted into a recipient. A recellularized liver as provided herein to be transplanted as a functional organ. In some cases, function can be determined through patency of the vasculature of the organ for a prolonged period of time. Patency can be measured using, for example, the methods described in the examples below. For instance, a graft can be connected to a peristaltic pump and subjected to physiologically achievable venous pressure. In some cases, a pressure of between 5 to 50 mm Hg can be utilized to determine patency through the venous vasculature or 40-120 mm Hg through the arterial vasculature.

In some aspects, a lifespan of a subject may be extended after transplantation of a composition, such as an organ or portion thereof provided herein. For example, a lifespan of a subject may be extended from about 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 15 years, 20 years, 30 years, 40 years, 50 years, 60 years, 70 years, 80 years, 90 years, or up to about 100 years after transplantation. In some aspects, transplantation of a composition, such as an organ or portion thereof provided herein, may reduce the need of a secondary treatment in a subject. Secondary treatments can refer to dialysis, pacemakers, respirators, and combinations thereof.

### In-Vitro Screening Assays and Filtering

Decellularized and recellularized organs or portions thereof provided herein can also be used in vitro to screen a wide variety of compounds, for effectiveness and cytotoxicity of pharmaceutical agents, chemical agents, growth/regulatory factors. The cultures can be maintained in vitro and exposed to the compound to be tested. The activity of a cytotoxic compound can be measured by its ability to damage or kill cells in culture. This may readily be assessed by vital staining techniques. The effect of growth/regulatory factors may be assessed by analyzing the cellular content of the matrix, e.g., by total cell counts, and differential cell counts. This may be accomplished using standard cytological and/or histological techniques including the use of immunocytochemical techniques employing antibodies that define type-specific cellular antigens. The effect of various drugs on normal cells cultured in the reconstructed artificial organs may be assessed.

Decellularized and recellularized organs or portions thereof provided herein can be used in vitro to filter aqueous solutions, for example, a reconstructed artificial kidney may be used to filter blood. Using the reconstructed kidney provides a system with morphological features that resemble the in vivo kidney products. This system may be suitable for hemodialysis. In some aspects, the system may also be useful for hemofiltration to remove water and low molecular weight solutes from blood. The artificial kidney may be maintained in-vitro and exposed to blood which may be infused into the luminal side of the artificial kidney. The processed aqueous solution may be collected from the abluminal side of the engineered kidney. The efficiency of filtration may be assessed by measuring the ion, or metabolic waste content of the filtered and unfiltered blood.

### Delivery Vehicle

Decellularized and recellularized organs or portions thereof provided herein can be used as a vehicle for introducing genes and gene products in vivo to assist or improve the results of the transplantation and/or for use in gene therapies. For example, cultured cells, such as endothelial cells, can be engineered to express gene products. The cells can be engineered to express gene products transiently and/or under inducible control or as a chimeric fusion protein anchored to the endothelial cells, for example, a chimeric molecule composed of an intracellular and/or transmembrane domain of a receptor or receptor-like molecule, fused to the gene product as the extracellular domain. In another embodiment, the endothelial cells can be genetically engineered to express a gene for which a patient is deficient, or which would exert a therapeutic effect. The genes of interest engineered into the endothelial cells or parenchyma cells need to be related to the disease being treated. For example, for a kidney disorder, the endothelial or cultured kidney cells can be engineered to express gene products that would ameliorate the kidney disorder.

In addition, the decellularized and recellularized organs or portions thereof provided herein can be used to make recombinant proteins, for example, alpha-1 antitrypsin (A1AT) or fibrinogen. The recombinant proteins provided herein can be isolated for use in the treatment of a disease.

### Combination Therapy

In some cases, a recellularized liver can be transplanted or used in an ex-vivo circuit along with systemic administration of an immunosuppressor. In some embodiments, administration of an immunosuppressor prolongs patency of a transplanted organ. In some cases, an immunosuppressor can be a corticosteroid, a Janus kinase inhibitor, a calcineurin inhibitor, an mTOR inhibitor, an IMDH inhibitor, a biologic, a monoclonal antibody, or any combination thereof. Examples of corticosteroids can include prednisone, budesonide, prednisolone, and methylprednisolone. Examples of Janus kinase inhibitors can include tofacitinib. Examples of calcineurin inhibitors can include cyclosporine and tacrolimus. Examples of mTOR inhibitors can include sirolimus and everolimus. Examples of IMDH inhibitors can include azathioprine, leflunomide, and mycophenolate. Examples of immunosuppressive biologics can include abatacept, adalimumab, anakinra, certolizumab, etanercept, golimumab, infliximab, ixekizumab, natalizumab, rituximab, secukinumab, tocilizumab, ustekinumab, and vedolizumab. Examples of immunosuppressive monoclonal antibodies can include basiliximab and daclizumab. Such immunosuppressors can be administered to a recipient of a recellularized liver via enteral routes (including oral, gastric or duodenal feeding tube, rectal suppository and rectal enema), parenteral routes (injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal or topical (including epicutaneous, dermal, enema, eye drops, ear drops, intranasal, vaginal) administration. Immunosuppressors can be administered to a recipient at a dose of from about 1 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg.

Other embodiments and used of the invention will be apparent to those skilled in the art from consideration of the specification and practice of the invention provided herein. All U.S. patents and other references noted herein for whatever reason are specifically incorporated by reference. The specification and examples should be considered exemplary only with the true scope and spirit of the invention indicated by the following claims.

### Exemplary Embodiments

Provided herein are methods of making an at least partially recellularized organ composition, including but not limited to liver, kidney, lung and heart, wherein the methods comprise: (a) treating a non-human animal organ with an anti-viral treatment; (b) perfusion decellularizing the non-human animal organ to obtain a decellularized extracellular matrix; (c) contacting the decellularized extracellular matrix with a cell composition comprising a population of human cells to form an at least partially recellularized organ composition. Provided herein are methods of making an at least partially recellularized liver composition, wherein the methods comprise: (a) treating a non-human animal liver with an anti-viral treatment; (b) perfusion decellularizing the non-human animal liver to obtain a decellularized extracellular matrix; (c) contacting the decellularized extracellular matrix with a cell composition comprising a population of human liver cells to form an at least partially recellularized liver composition. Further provided herein are methods, wherein the anti-viral treatment comprises irradiation of the non-human animal liver with an electron beam (E-beam). Further provided herein are methods, wherein the irradiation comprises exposing the non-human animal liver to an electron beam dose that is from about 2 kGy to about 50 kGy. Further provided herein are methods, wherein the irradiation comprises exposing the non-human animal liver to an electron beam dose that is from about 5 kGy to about 25 kGy. Further provided herein are methods, wherein the irradiation comprises exposing the non-human animal liver to an electron beam dose that is from about 10 kGy to 20 kGy. Further provided herein are methods, further comprising contacting the decellularized extracellular matrix with a peroxy acid or hydrogen peroxide. Further provided herein are methods, wherein the anti-viral treatment comprises irradiation of the non-human animal liver with an electron beam (E-beam), and wherein the method further comprises contacting the decellularized extracellular matrix with a peroxy acid or hydrogen peroxide. Further provided herein are methods, wherein the peroxy acid comprises peroxyacetic acid, peracetic acid, peroxycarboxylic acid, derivatives, or combinations thereof. Further provided herein are methods, further comprising contacting the decellularized extracellular matrix with an additional cell composition comprising a population of human vascular endothelial cells (HUVECs). Further provided herein are methods, wherein the cell composition comprises both a population of human liver cells and a population of HUVECs. Further provided herein are methods, wherein the population of human liver cells are primary human liver cells. Further provided herein are methods, wherein the population of human liver cells are in vitro-differentiated human liver cells. Further provided herein are methods, wherein the in vitro-differentiated human liver cells are derived from or differentiated from a population of embryonic stem cells, a population of induced pluripotent stem cells (iPSCs), or a population of adult stem cells. Further provided herein are methods, wherein the population of human liver cells are contacted with a protease prior to contacting the decellularized extracellular matrix with the cell composition in step (c). Further provided herein are methods, wherein the at least partially recellularized liver is characterized as having an increase in the level of ammonia clearance relative to a population of liver cells that are not engrafted onto a decellularized extracellular matrix. Further provided herein are methods, wherein the non-human animal is a non-human mammal. Further provided herein are methods, wherein the non-human mammal is an ungulate. Further provided herein are methods, wherein the ungulate is a pig. Further provided herein are methods, wherein the non-human animal liver is frozen prior to the anti-viral treatment.

Provided herein are methods of making an at least partially recellularized organ composition, wherein the methods comprise: (a) treating a non-human animal organ with an anti-viral treatment; (b) perfusion decellularizing the non-human animal organ to obtain a decellularized extracellular matrix; (c) contacting the decellularized extracellular matrix with a cell composition comprising a population of human cells to form an at least partially recellularized organ composition. Further provided herein are methods, wherein the anti-viral treatment comprises irradiation of the non-human animal organ with an electron beam (E-beam). Further provided herein are methods, wherein the irradiation comprises exposing the non-human animal organ to an electron beam dose that is from about 2 kGy to about 50 kGy. Further provided herein are methods, wherein the irradiation comprises exposing the non-human animal organ to an electron beam dose that is from about 5 kGy to about 25 kGy. Further provided herein are methods, wherein the irradiation comprises exposing the non-human animal organ to an electron beam dose that is from about 10 kGy to 20 kGy. Further provided herein are methods, further comprising contacting the decellularized extracellular matrix with at least one of a peroxy acid or hydrogen peroxide. Further provided herein are methods, wherein the anti-viral treatment comprises irradiation of the non-human animal organ with an electron beam (E-beam), and wherein the method further comprises contacting the decellularized extracellular matrix with at least one of a peroxy acid or hydrogen peroxide. Further provided herein are methods, wherein the peroxy acid comprises peroxyacetic acid, peracetic acid, peroxycarboxylic acid, derivatives, or combinations thereof. Further provided herein are methods, further comprising contacting the decellularized extracellular matrix with an additional cell composition comprising a population of human vascular endothelial cells (HUVECs). Further provided herein are methods, wherein the cell composition comprises both a population of human organ cells and a population of HUVECs. Further provided herein are methods, wherein the population of human cells comprise human liver cells, human kidney cells, human lung cells, human heart cells, or any other cells of a human solid organ. Further provided herein are methods, wherein the population of human cells are in vitro-differentiated human cells. Further provided herein are methods, wherein the in vitro-differentiated human cells are differentiated from a population of embryonic stem cells, a population of induced pluripotent stem cells (iPSCs), or a population of adult stem cells. Further provided herein are methods, wherein the population of human cells are contacted with a protease prior to contacting the decellularized extracellular matrix with the cell composition in step (c). Further provided herein are methods, wherein the at least partially recellularized organ is characterized as having an increase in a cellular activity relative to a cellular activity of population of human cells that are not engrafted onto a decellularized extracellular matrix. Further provided herein are methods, wherein the non-human animal is a non-human mammal. Further provided herein are methods, wherein the non-human mammal is an ungulate. Further provided herein are methods, wherein the ungulate is a pig. Further provided herein are methods, wherein the non-human animal organ is frozen prior to the anti-viral treatment.

Provided herein are methods of making an at least partially recellularized organ composition, the methods comprising: (a) treating a non-human animal organ with an anti-viral treatment; (b) perfusion decellularizing the non-human animal organ to obtain a decellularized extracellular matrix; (c) contacting the decellularized extracellular matrix with a first cell composition comprising a population of human vascular endothelial cells; and (d) contacting the decellularized extracellular matrix with a second cell composition to form an at least partially recellularized organ composition.

Provided herein are methods of making an at least partially recellularized liver composition, the methods comprising: (a) treating a non-human animal liver with an anti-viral treatment; (b) perfusion decellularizing the non-human animal liver to obtain a decellularized extracellular matrix; (c) contacting the decellularized extracellular matrix with a first cell composition comprising a population of human vascular endothelial cells; and (d) contacting the decellularized extracellular matrix with a second cell composition comprising a population of human liver cells to form an at least partially recellularized liver composition. Further provided herein are methods, wherein the decellularized extracellular matrix is contacted with the second cell composition when the first cell composition is characterized as having a glucose consumption rate of at least about 10 mg/hr. Further provided herein are methods, wherein the decellularized extracellular matrix is contacted with the second cell composition when the first cell composition is characterized as having a glucose consumption rate of at least about 20 mg/hr. Further provided herein are methods, wherein the decellularized extracellular matrix is contacted with the second cell composition when the first cell composition is characterized as having a glucose consumption rate of at least about 30 mg/hr. Further provided herein are methods, wherein the decellularized extracellular matrix is contacted with the second cell composition when the first cell composition is characterized as having a glucose consumption rate of at least about 40 mg/hr. Further provided herein are methods, wherein the decellularized extracellular matrix is contacted with the second cell composition when the first cell composition is characterized as having a glucose consumption rate of at least about 50 mg/hr. Further provided herein are methods, wherein the decellularized extracellular matrix is contacted with the second cell composition when the first cell composition is characterized as having a glucose consumption rate of at least about 60 mg/hr. Further provided herein are methods, wherein the decellularized extracellular matrix is contacted with the second cell composition when the first cell composition is characterized as having a glucose consumption rate of at least about 70 mg/hr. Further provided herein are methods, wherein the decellularized extracellular matrix is contacted with the second cell composition when the first cell composition is characterized as having a glucose consumption rate of at least about 80 mg/hr. Further provided herein are methods, wherein the decellularized extracellular matrix is contacted with the second cell composition when the first cell composition is characterized as having a glucose consumption rate of at least about 90 mg/hr. Further provided herein are methods, wherein the decellularized extracellular matrix is contacted with the second cell composition when the first cell composition is characterized as having a glucose consumption rate of at least about 100 mg/hr. Further provided herein are methods, wherein the decellularized extracellular matrix is contacted with the second cell composition at least 10 days after contacting the decellularized extracellular matrix with the first cell composition. Further provided herein are methods, wherein the decellularized extracellular matrix is contacted with the second cell composition at least 12 days after contacting the decellularized extracellular matrix with the first cell composition. Further provided herein are methods, wherein the decellularized extracellular matrix is contacted with the second cell composition between 10 and 20 days after contacting the decellularized extracellular matrix with the first cell composition. Further provided herein are methods, wherein the at least partially recellularized liver composition is characterized as having an increase in a level of ammonia clearance relative to a population of liver cells that are not in the form of an at least partially recellularized liver composition. Further provided herein are methods, wherein the anti-viral treatment comprises irradiation of the decellularized extracellular matrix with an electron beam (E-beam). Further provided herein are methods, wherein the irradiation comprises exposing the non-human animal liver to an electron beam dose that is from about 2 kGy to about 50 kGy. Further provided herein are methods, wherein the irradiation comprises exposing the non-human animal liver to an electron beam dose that is from about 5 kGy to about 25 kGy. Further provided herein are methods, wherein the irradiation comprises exposing the non-human animal liver to an electron beam dose that is from about 10 kGy to 20 kGy. Further provided herein are methods, further comprising contacting the decellularized extracellular matrix with a peroxy acid or hydrogen peroxide. Further provided herein are methods, wherein the peroxy acid comprises peroxyacetic acid, peracetic acid, peroxycarboxylic acid, derivatives, or combinations thereof. Further provided herein are methods, wherein the population of human liver cells are primary human liver cells. Further provided herein are methods, wherein the population of human liver cells are in vitro-differentiated human liver cells. Further provided herein are methods, wherein the in vitro-differentiated human liver cells are derived from or differentiated from a population of embryonic stem cells, a population of induced pluripotent stem cells (iPSCs), or adult stem cells. Further provided herein are methods, wherein the population of human liver cells are contacted with a protease prior to contacting the decellularized extracellular matrix with the second cell composition in step (d). Further provided herein are methods, wherein the at least partially recellularized liver composition is characterized as having an increase in the level of ammonia clearance relative to a population of liver cells that are not in the form of an at least partially recellularized liver composition. Further provided herein are methods, wherein the non-human animal is a non-human mammal. Further provided herein are methods, wherein the non-human mammal is an ungulate. Further provided herein are methods, wherein the ungulate is a pig. Further provided herein are methods, wherein the non-human animal liver is frozen prior to the anti-viral treatment.

Provided herein are compositions comprising an at least partially recellularized organ produce by any of the methods provided herein.

Provided herein are compositions comprising an at least partially recellularized liver composition produced by any of the methods provided herein. Provided herein are at least partially recellularized livers comprising: (a) a porcine extracellular matrix; (b) a population of human endothelial cells and a population of human liver cells engrafted onto the porcine extracellular matrix, wherein the at least partially recellularized livers have an increase in ammonia clearance relative to a population of porcine liver cells engrafted onto a porcine extracellular matrix. Provided herein are at least partially recellularized livers comprising: (a) a microbial particle diminished, perfusion-decellularized porcine extracellular matrix; (b) a population of human endothelial cells and a population of human liver cells engrafted onto the porcine extracellular matrix, wherein the at least partially recellularized livers have an increase in ammonia clearance relative to a population of porcine liver cells engrafted onto a porcine extracellular matrix. Further provided herein are at least partially recellularized livers, wherein the at least partially recellularized liver comprises one or more surface marker selected from the group consisting of: a cluster of differentiation (CD) 31, CD105, and Asialoglycoprotein receptor 1 (ASGR1). Further provided herein are at least partially recellularized livers, wherein the at least partially recellularized liver is characterized as having an increase in a level of alpha-1-antitrypsin (A1AT) relative to a population of liver cells that are not engrafted onto a porcine extracellular matrix. Further provided herein are at least partially recellularized livers, wherein the at least partially recellularized liver is characterized as having an increase in the level of fibrinogen relative to a population of liver cells that are not engrafted onto a porcine extracellular matrix. Further provided herein are at least partially recellularized livers, wherein the at least partially recellularized liver is characterized as having an increase in the level of fibrinogen relative to a population of porcine liver cells that are engrafted onto a porcine extracellular matrix. Further provided herein are at least partially recellularized livers, wherein the at least partially recellularized liver is characterized as having an increase in the level of ammonia clearance relative to a population of human liver cells that are not engrafted onto a porcine extracellular matrix. Further provided herein are at least partially recellularized livers, wherein the at least partially recellularized liver is characterized as having ammonia clearance following cold storage, wherein the cold storage is for a period of at least 6 hours or more. Further provided herein are at least partially recellularized livers, wherein the at least partially recellularized liver is characterized as having urea production following cold storage, wherein the cold storage is for a period of at least 6 hours or more. Further provided herein are at least partially recellularized livers, further comprising an anti-microbial agent. Further provided herein are at least partially recellularized livers, wherein the anti-microbial agent is an anti-viral agent. Further provided herein are at least partially recellularized livers, wherein the anti-viral agent comprises peroxyacetic acid. Further provided herein are at least partially recellularized livers, wherein the population of human liver cells are primary human liver cells. Further provided herein are at least partially recellularized livers, wherein the population of human liver cells are in vitro-differentiated human liver cells. Further provided herein are at least partially recellularized livers, wherein the in vitro-differentiated human liver cells are derived from or differentiated from a population of embryonic stem cells, a population of induced pluripotent stem cells (iPSCs), or adult stem cells.

Provided herein are compositions, wherein the compositions comprise: the at least partially recellularized liver provided herein; and an extracellular matrix protein or solution. Further provided herein are compositions, wherein the compositions further comprise cell culture medium.

Provided herein are ex-vivo methods of treating a liver disease in a subject, wherein the methods comprise: producing a blood circuit, wherein the blood circuit comprises blood from the subject in fluid communication with an at least partially recellularized liver provided herein, wherein the at least partially recellularized liver filters blood from the subject and clears ammonia, thereby treating the liver disease in the subject.

Provided herein are methods for treating a liver disease in a subject, wherein the methods comprise: administering to a subject the at least partially recellularized liver provided herein, thereby treating the liver disease in the subject. Further provided herein are methods, wherein the liver disease comprises acute liver failure (ALF).

### EXAMPLES

### Example 1. Porcine Organ Procurement and Whole Liver Decellularization

Whole porcine livers (500 to 700 grams) were excised from cadaveric pigs, rinsed with PBS, flushed with saline, and frozen. Frozen livers were treated with an electron beam (E-Beam) prior to decellularization (E-Beam Services, Cranbury, NJ). In preparation for decellularization, the porcine livers were thawed at room temperature and the Portal Vein (PV), Suprahepatic Inferior Vena Cava (sIVC), and Inferior Vena Cava (IVC) were cannulated. The cannulated livers underwent perfusion decellularization with 1% Triton X-100 (Amresco, M143), and 0.6% sodium dodecyl sulfate (Amresco, 0227) through the PV and sIVC at target pressures between 12 to 17 mmHg. The decellularized livers were then disinfected with peracetic acid (PAA; U.S. Water, BI0032-6). The decellularized grafts were washed with phosphate buffered saline (PBS; Corning 21-040-CMX12) and stored. The decellularization process was completed in an ISO 7 cleanroom.

### Example 2: Residuals Testing

Punch biopsies from fully decellularized liver grafts were collected, weighed and digested prior to assays for SDS, Triton and DNA residuals. 80 mg samples for SDS and triton residuals were digested in 15 uL of 20 mg/mL Proteinase K (Qiagen), vortexed and heated at 60° C. for 18-24 hours. Samples were then assayed for any detectable SDS and Triton through Pace Analytical (Minneapolis MN). Punch biopsies for DNA testing and known standards (Thermo Fisher Scientific) were digested with 40 uL of 20 mg/mL Proteinase K (Thermo Fisher Scientific) and then measured on a Qubit 3.0 Fluorometer (Thermo Fisher Scientific).

### Example 3: Evaluation of E-Beam Irradiation Step on Viral Inactivation

Evaluation of the effectiveness of E-Beam as a method of viral inactivation was performed through Charles River Laboratory Services (Wayne, PA) in compliance with the U.S. Food and Drug Administration's Good Manufacturing Practice regulations as found in Title 21 CFR parts 210 and 211 with no deviations from the test methods that impacted the quality of integrity of the test results. In brief, liver tissues were sectioned into 1″×2″ patches and rinsed with 100 mL 0.9% saline, and incubated with 1 mL of virus, either Murine Leukemia Virus (MuLV), Psuedorabies (PRV), Reoviridae (Reo3) and Porcine parovirus (PPV), for 15 minutes. After viral spiking the materials were treated with E-Beam radiation (2 aliquots per virus per irradiation run) at either 10 kGy or 20 kGy then sampled and analyzed for viral titer. Reference runs were treated identically but omitted the E-Beam treatment.

### Example 4: Huvec Cell Culture and Seeding of Decellularized Liver Scaffolds

Human umbilical vein endothelial cells (HUVECS) (Lonza, Cat #C2519A) were cultured at 37° C. and 5% C02 in antibiotic-free Endothelial Cell Growth media (R&D Systems, CCM027) supplemented with 2% fetal bovine serum (Corning), 50 mg/L ascorbic acid (Sigma), 1 mg/L hydrocortisone (Sigma), 20 μg/L FGF (R&D Systems, MN), 5 μg/L VEGF (R&D Systems), 15 μg/L R³IGF (Sigma), 1000 U/L heparin (Sigma), and 1.5 μM acetic acid (Sigma). Cells were harvested with 0.25% trypsin-EDTA (ThermoFisher) at 90-100% confluency. Decellularized porcine livers were mounted in bioreactors and perfused with antibiotic free media (37° C., 5% CO₂) for 72 h to confirm the absence of microbial contamination. HUVECs collected at passage 5-9 were infused through the sIVC with a syringe (1.60×10⁸) cells in 180 mL culture media. Extracorporeal bioengineered liver (BEL) grafts then underwent a 1-hour static culture to allow for cell attachment within the scaffold. During this 1-hour static hold, temperature and dissolved gas concentrations were maintained in the bioreactor by perfusing culture media through the system while bypassing the organ. After the 1-hour static hold, 40×10⁶ HUVECs in 90 mL culture media were infused into the sIVC with a syringe under continuous flow at 300 mL/min. After an overnight culture, the organ was manually flipped to perfuse on the portal vein. HUVECs were collected and seeded through the portal vein in the same manner described above for the sIVC. Following seeding, culture media was replaced daily, and volumes were continually adjusted to ensure that glucose levels remained above 0.2 g/L within a 24 h period. Media perfusion into the scaffold was maintained at a maximum 300 mL/min flow rate, with maximum pressures at 30 mmHg.

### Example 5: Porcine Hepatocyte Isolation

Fresh whole livers (450-1000 g) harvested from pigs and primary porcine liver cells were isolated as previously described (Anderson B A et. al., 2021). This study was reviewed and approved (No. 040420) by the Institutional Animal Care and Use Committee (IACUC) for MRS/Collagen Solutions. Cell viability and yield were quantified by trypan blue dye exclusion on a hemocytometer. Final cell pellet was resuspended in 1 L of University of Wisconsin (UW) Belzer solution (Bridge to life, Northbrook, IL).

### Example 6: Human Hepatocyte Isolation

Organs for research to advance medical science were obtained by Donor Network West, NDRI, and Southwest Transplant Alliance, and were generously gifted with the consent of the donor or the donor's next of kin. Liver tissue was flushed via the inferior vena cava, hepatic artery, and portal vein using Lactated Ringers Solution (LRS; Hutchins & Hutchins, VA). The right and left lobes were resected and exposed vessels flushed with cold LRS and cannulated for perfusion. Liver tissue was perfused (10-50 mL/min) with Liver Perfusion Solution I (VitroPrep, NC) for 12-15 minutes followed by perfusion with Liver Perfusion Solution II (VitroPrep, NC) for 20-40 minutes. Liver Perfusion Solution II was supplemented with both collagenase MA (3 mg/L; Vitacyte) and protease BP (2.5 mg/L; Vitacyte) to initiate digestion. Solutions were not recirculated. Digested tissue was diluted in human hepatocyte isolation medium (HHIM; DMEM with 10% FBS) and passed through a sieve set with the following sizes (1 mm-optional, 500, 250, 90 μm). Hepatocytes were enriched by centrifugation (110×g, room temperature, 10 min) and washed once with HHIM. Cell viability and yield were quantified by trypan blue dye exclusion on a hemocytometer. The final cell pellet was resuspended in 1 L of UW solution at a concentration of ≤5 million cells per mL.

### Example 7: Flow Cytometry

HUVECs were taken from final cell suspension prior to seeding into grafts. Cells were fixed using 2% paraformaldehyde (Electron Microscopy Sciences) and stained for CD31 (BIORAD, #MCA1738; 1:100 dilution) and CD105 (BIORAD, #MCA1557; 1:100 dilution) expression. Expression was evaluated by flow cytometry using a BD Accuri C6 Flow Cytometer (BD Biosciences). Data analysis was done using FlowJo software.

Primary human liver cell (PHLC) samples were taken from the final cell suspension of freshly isolated PHLCs. Cells were fixed using 4% paraformaldehyde (Electron Microscopy Sciences), permeabilized with Triton X-100 (Sigma) and stored in FACS buffer (1×PBS (Corning) with 10% BSA (Sigma) and 0.5% NaN₃(Ricca)). Cells were stained for ASGR1 expression (antibody from R&D Systems, #FAB43941R; 1:20 dilution) and analyzed by flow cytometry using an Attune NxT Flow Cytometer (Thermo Fisher Scientific). Hepatocytes were identified via size using forward and side scatter and ASGR1 expression as compared to the isotype control (R&D Systems; IC002R; 1:20 dilution). Data analysis was done using FlowJo software.

### Example 8: Hepatocyte Seeding of Extracorporeal Bioengineered Liver (Bel) Scaffolds

Hepatocytes from either porcine or human were allowed to pellet overnight at 4° C. Following the overnight hold, the supernatant was removed from the pellet and either 10×10⁹ porcine hepatocytes or 5×10⁹ human hepatocytes were diluted into Williams' E medium (Gibco) supplemented with 2% fetal bovine serum (Corning), 50 mg/L ascorbic acid (Sigma), 20 μg/L FGF (R&D systems), 5 μg/L VEGF (R&D Systems), 5 μg/L EGF (R&D Systems), 1000 U/L heparin (Sigma), 3 g/L human albumin (CSL, Behring), 150 μg/L Linoleic Acid (Sigma), 0.1 μM dexamethasone (Sigma), 40 μg/L human glucagon (Novalis), 6 mg/L human holo-transferrin (Sigma), 20 μg/L Gly-His-Lys (Sigma), 5 ug/L sodium selenite, 1 g/L L-carnitine (Sigma), 0.2 g/L-arginine (Sigma), and 10 mg/L glycine (Sigma). Porcine hepatocytes were diluted in the above media supplemented with 50 μg/L LONG® Arginine₃ Insulin-Like Growth Factor (IGF) and human hepatocytes were diluted in the above media supplemented with 8.4 U/L human insulin (Novolin). Both human and porcine hepatocytes were infused through the sIVC of the reendothelialized BEL scaffold perfusing at 350 mL/min (typically 12-16 days following the first HUVEC seeding) through repeated syringe injection at a rate of 94 mL/min. Hepatocyte-seeded BELs were then returned to continuous media perfusion through the PV with co-culture media at a maximum flow rate of 300 mL/min, and maximum pressures of 30 mmHg.

### Example 9: Analysis of Metabolites

Media Samples from bioreactors were collected daily and immediately assayed on a CEDEX Bio HT bioanalyzer (Roche) to determine levels of glucose, ammonia, and lactate dehydrogenase activity in the culture media. Measured glucose concentrations were used to calculate daily consumption rates over a 24 h period prior to complete media change and used to determine the level of re-endothelialization and necessary media volume for the following media change. Additional samples were collected daily for quantification of urea, A1AT, and fibrinogen and stored at −80° C. for analysis. Urea levels were analyzed via commercially available kits with minor modifications to manufacturer guidance (BioAssay Systems, DIUR-100). A1AT quantification was performed with a commercially available kit with minor modifications (abcam, ab189579). Fibrinogen was assessed with minor modifications to the commercially available kit from abcam (ab241383).

### Example 10: Sterility Testing

Due to the short time between production of HUVECs and PHLCs and their introduction into the manufacturing process, microbial testing was focused on detecting any contaminants introduced by these starting materials after they are seeded into the decellularized matrix. USP <71> compendial sterility testing was performed post-HUVEC seeding and after transfer for transport, where interim 5 to 7-day reads were evaluated prior to 14-day results. A rapid microbial method (RMM) using a Gram stain test was also performed to aid in real-time detection of microbial contamination. Rapid mycoplasma testing via nucleic acid amplification was performed post-HUVEC seeding and prior to cold storage simulation. Qualification of the method demonstrated a limit of detection aligning with European Pharmacopoeia 2.6.7 with suitable comparability to the culture based USP<63> test methodologies.

### Example 11: Ammonia Clearance and Urea Production Assays

Sixteen to twenty hours after seeding hepatocytes, the bioreactors containing the bi-culture BELs were media changed into fresh media. As media was perfused through the system while bypassing the organ, ammonium chloride was dosed into the system at a final concentration of 200 μM. The media was allowed to circulate with the dosed ammonium chloride for 10 minutes before a 0-hour sample was collected, and the organ opened to ammonium chloride-containing media to resume normal media perfusion. Samples were collected after an hour and the ammonia levels quantified on the CEDEX Bio HT Bioanalyzer.

### Example 12: Data Analysis

Ammonia clearance data was collected in real time. Fibrinogen, alpha-1 antitrypsin, and urea production data were collected and analyzed from frozen samples and analyzed post-hoc for statistical significance. AC rates were calculated by measuring the difference in ammonia levels between the 0- and 1-hour post-bolus time points. All data comparisons were applied using a one-way ANOVA (α<0.05 deemed significant) between days 1-7, 1-3, and 5-7. A1AT data generated from days 6 and 7 was not included due to a low “N”. Data was reported as mean and one standard deviation where applicable.

### Example 13: Cold Storage Simulation

For the cold storage study, BEL grafts were removed from bioreactor perfusion after 72 hours of culture and transferred to a static transport container. Grafts were flushed with 2 L of cold Belzer UW solution at 300 mL/min. Transport containers were stored in a cooler filled with ice for 14-16 hrs. After cold storage, BELs were flushed with 2 L of room temperature PBS at 300 mL/min and returned to the bioreactor system where perfusion was restarted with 4 L of fresh culture media. Ammonia clearance was evaluated approximately 10 min after media perfusion began. Samples were collected for A1AT and fibrinogen quantification after approximately 24 hours of media perfusion. Ammonia clearance was evaluated at this timepoint as well.

### Example 14: Blood Perfusion Studies

For the in vitro blood perfusion studies, each BEL was connected to a circuit consisting of silicone tubing, a pressure transducer (Deltran, DPT-100), and a peristaltic pump (Cole-Palmer, 07522-20), and an oxygenator (LivaNova, 050703) warmed with a recirculating water bath to 37° C. and receiving 20% O₂, 5% CO₂, and 75% N₂ blended gas. Freshly collected heparinized porcine blood was warmed to 37° C. and the activated clotting time (ACT) was measured (ITC, Hemochron Response). A solution of protamine sulfate was gradually added to the blood to neutralize heparin until an ACT of 400-600 was reached. Two liters of blood was introduced into the circuit and perfused through the portal vein of the BEL at an initial flow rate of 350 mL/min. Pressures were recorded over 180 minutes of blood perfusion.

### Example 15: E-Beam Irradiation Treatment Inactivates Xenotropic Viruses in Native Porcine Livers Prior to Decellularization

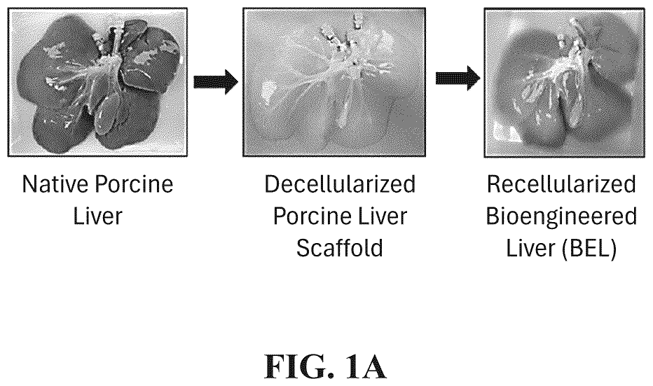

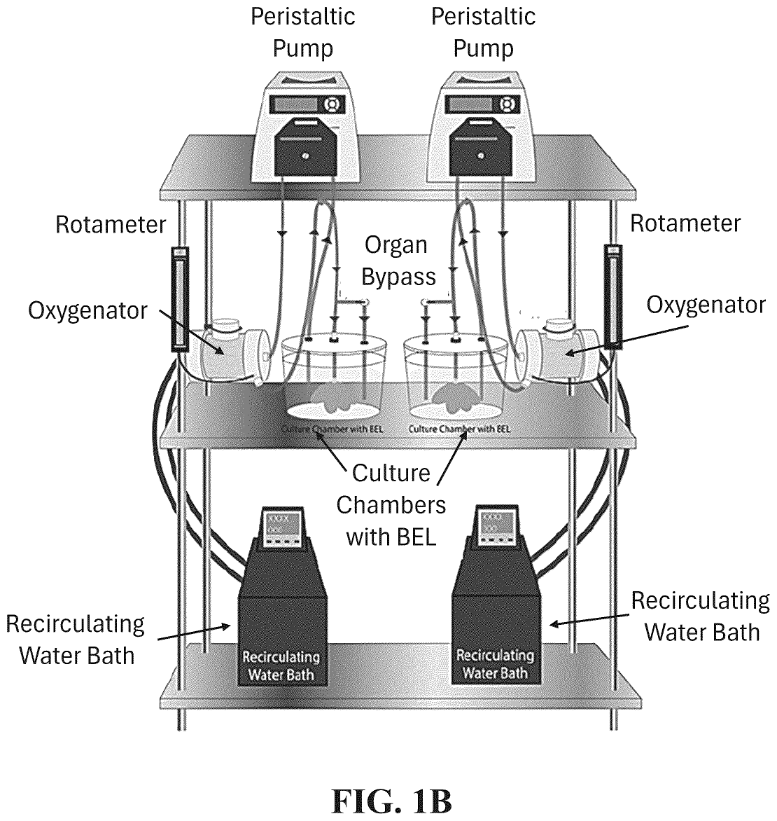

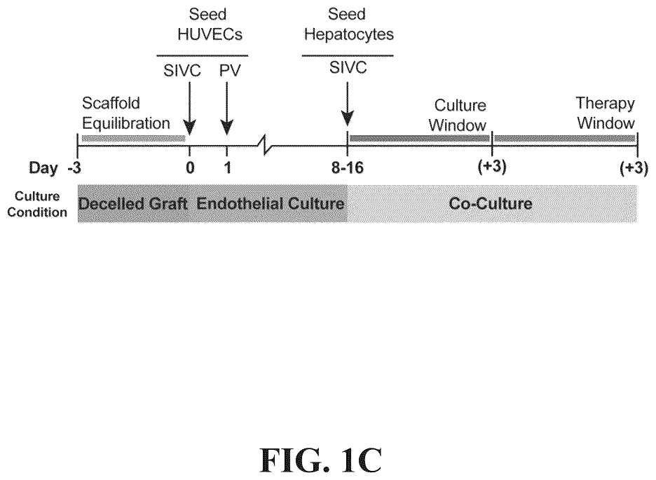

Whole porcine livers were collected from cadaveric pigs, decellularized, and recellularized with HUVECs and PHLCs as described above (FIG. 1A). To eliminate the risk of xenotropic virus transmission from a decellularized liver scaffold to a human patient, prior to decellularization process, all whole porcine livers are treated with E-Beam and PAA as a method of viral inactivation. Viral inoculations, performed through Charles River Laboratories (Wayne, PA), of Murine Leukemia Virus (MuLV), Psuedorabies (PRV), Reoviridae (Reo3) and Porcine parovirus (PPV) were spiked into porcine livers prior to treatment with either 10 kGy or 20 kGy E-Beam dose. While both 10 kGy and 20 kGy doses demonstrated a log reduction greater than 1 signifying viral inactivation, 20 kGy treatment had a greater effect on virus reduction and was chosen as standard treatment for all BELs (Table 2). To ensure no harmful residues remained as a byproduct of the decellularization process representative lots of decellularized porcine liver scaffolds were tested for triton, SDS, and DNA. All assayed grafts passed the desired acceptance criteria proving that the decellularization process successfully removes any harmful residuals prior to seeding with live cells (Table 3).

TABLE 2
E-Beam irradiation treatment inactivates xenotropic viruses
in native porcine livers prior to decellularization.

| | | | | | E-Beam Irradiation | | E-Beam Irradiation | |
|---|---|---|---|---|---|---|---|---|
| Virus Characteristics | | | | | (10 kGY) | | (20 kGY) | |
| Virus | Genome | Envelope | Size (nm) | Shape | Run 1 | Run 2 | Run 1 | Run 2 |
| Parvovirus | DNA | − | 18-24 | Icosahedral | 1.43 | 1.13 | 3.22 | 3.63 |
| (PPV) | | | | | | | | |
| Pseudorabies | DNA | + | 120-200 | Spherical | ≥3.56 | 4.35 | ≥3.68 | 3.69 |
| (PRV) | | | | | | | | |
| Reovirus 3 | RNA | − | 60-80 | Spherical | 5.55 | 5.59 | ≥5.19 | ≥5.61 |
| (Reo3) | | | | | | | | |
| Murine | RNA | + | 80-110 | Spherical | 1.52 | 1.4 | ≥4.52 | 4.75 |
| Leukemia | | | | | | | | |
| virus | | | | | | | | |
| (MuLV) | | | | | | | | |

TABLE 3
Representative Residual Testing of Decellularized Porcine Liver Scaffold.

| | | PL-ECM | | | | Calculated | Residual |
|---|---|---|---|---|---|---|---|
| | | amount | | | | residual SDS | DNA |
| PL-ECM | PL-ECM | used for | Total | Calculated | Total | (mg/20 g | (ng/mg |
| Batch | Tested | testing | Triton | Residual | SDS | tissue) | Tissue) |
| A | 1 | 76.8 | 116 | <1 | <LOD | <1 | <LOD |
| B | 2 | 73.6 | 102 | <1 | <LOD | <1 | <LOD |
| C | 3 | 82.0 | 49 | <1 | <LOD | <1 | <LOD |

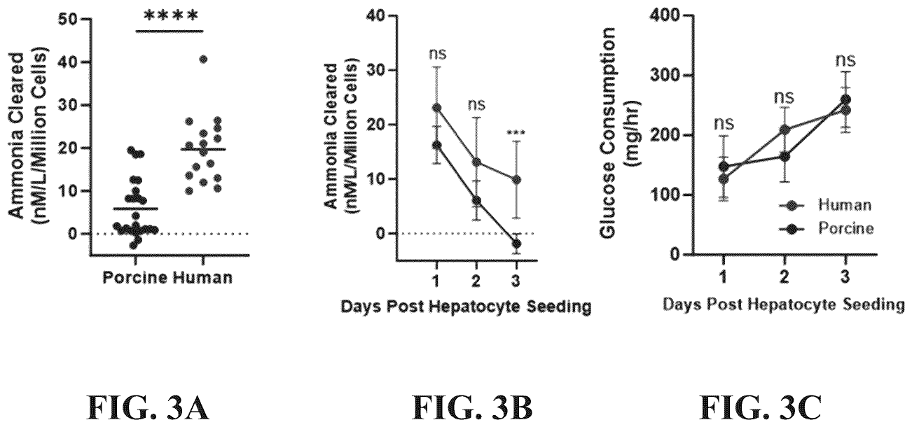

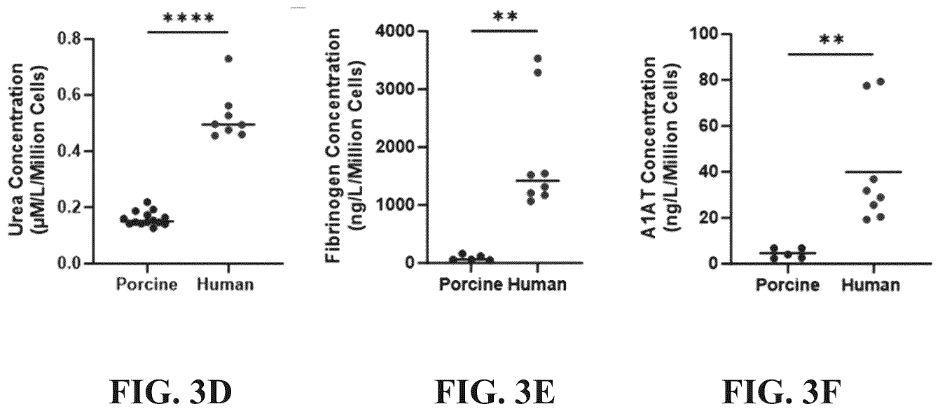

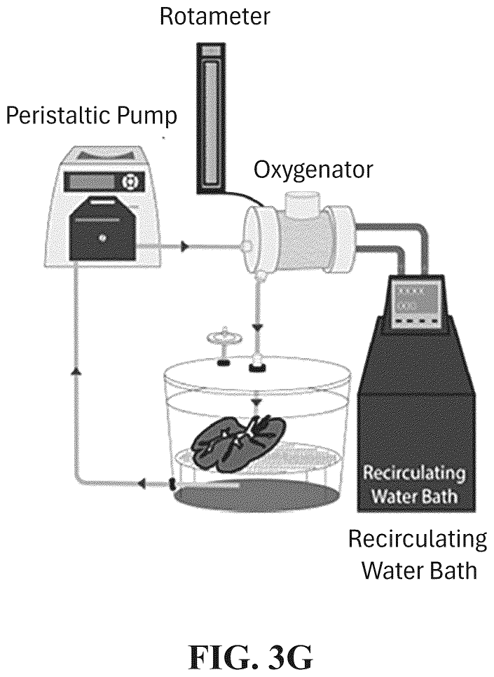

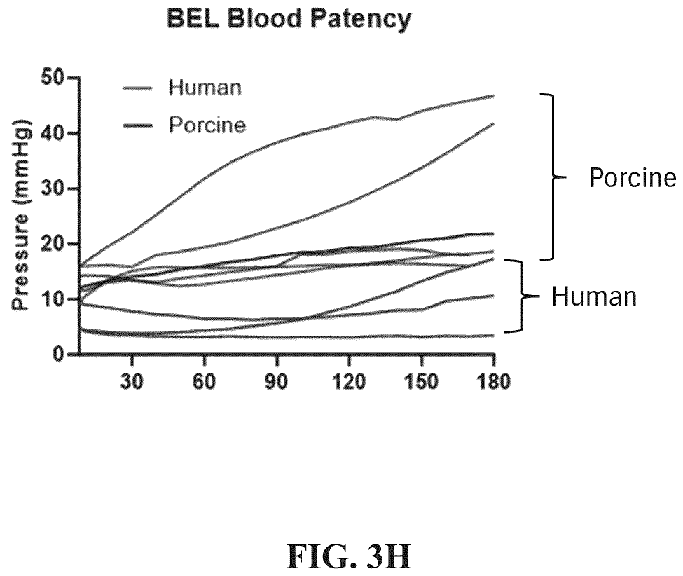

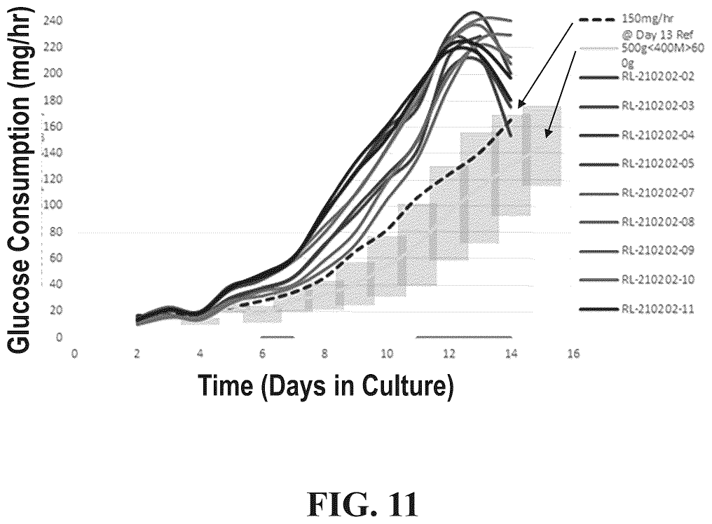

Following decellularization, scaffolds were installed into the bioreactor culture station (FIG. 11B) and perfused with media for a 3-day qualification period before endothelialization with HUVECs through the superior IVC and portal vein (FIG. 3C). These seeded scaffolds are cultured for eight to sixteen days to ensure complete HUVEC revascularization prior to primary hepatocyte seeding isolated from either human or porcine native livers (FIG. 1C). BELs were cultured for 3 days and then analyzed for function during the therapy window.

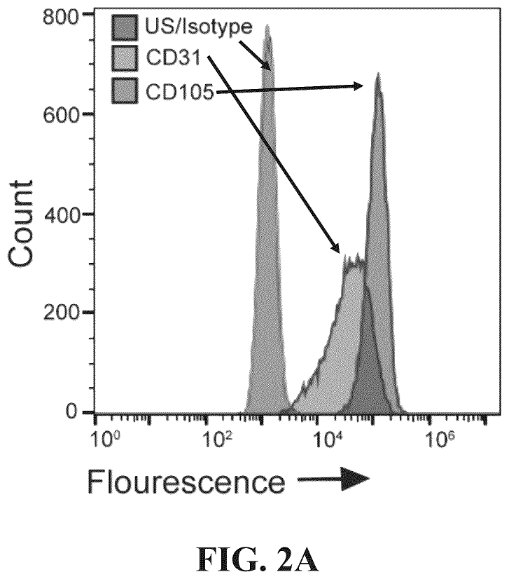

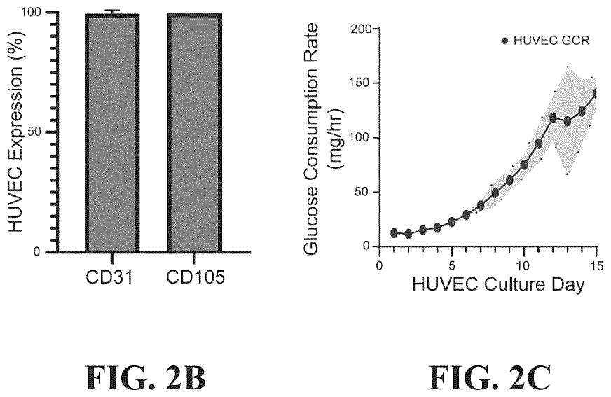

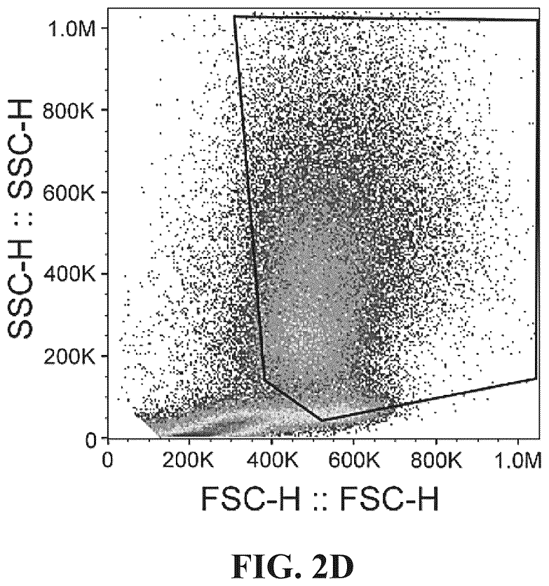

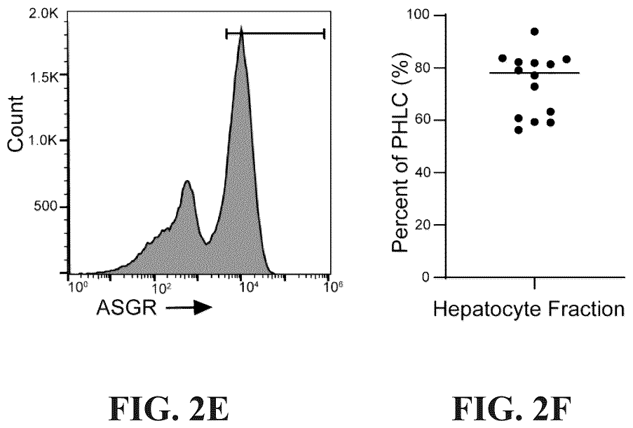

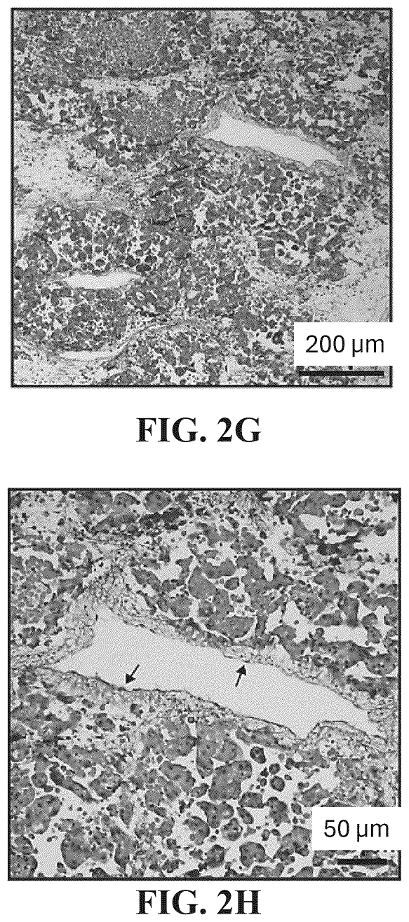

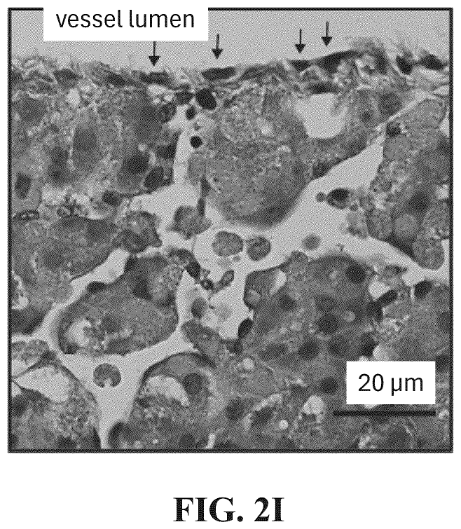

To confirm the phenotype of HUVECs prior to seeding into the decellularized porcine scaffold, the expression of endothelial specific markers CD31 and CD105 was assessed by flow cytometry (FIG. 2A). Both CD31 and CD105 are expressed in high levels in these cells. HUVEC expansion after seeding into the decellularized porcine scaffold is indicated by increasing glucose consumption over time (FIG. 2B), as well as histological examination of bi-culture BELs (FIG. 2C), where HUVECs are seen lining the vasculature.

PHLCs Flow cytometry was also used to evaluate the PHLC population after isolation. Hepatocyte phenotype was confirmed both by scatter profile (FIG. 2D) and expression of the hepatocyte-specific marker ASGR (FIG. 2E) After seeding, this hepatocyte population engrafts into the parenchyma of the revascularized liver scaffold (FIG. 2F-FIG. 2I).

To confirm the absence of any adventitious agents and contaminants, testing was performed post HUVEC seeding and again on day 14-16. All lots tested were suitable for use in our manufacturing process and determined to be free of microbial contamination, endotoxin and mycoplasma (Data not shown). Acquiring human hepatocytes presents several ethical considerations as well as logistical hurdles since the availability of donor livers is unpredictable. Therefore, our previously published work used primary porcine hepatocytes (Anderson B A et. al., 2021). However, to remove the immunological risks to patients commonly associated with xenotransplantation the inventors here have created BELs constructed entirely with human cell types. The function of these humanized BELs was assessed alongside porcine grafts manufactured under the same processes. Porcine and human BELs were subjected to an ammonia clearance assay the day after hepatocyte seeding. One hour after administration of a 200 μM bolus, human hepatocyte-seeded BELs cleared higher amounts of ammonia (96±31.5 μM of ammonia in comparison to 58.6±6.7 μM of ammonia by BELs seeded with porcine hepatocytes (FIG. 3A). Because a subset of porcine BELs was able to clear ammonia more than the human hepatocytes, the inventors wanted to compare the longevity of function to assess the potential for therapy. Even though some porcine BELs do, in fact, clear equal amount of ammonia on day 1 of culture, they do not retain this function by day three when compared to humanized BELs seeded with PHLCsa (FIG. 3B). The statistically significant increase in levels of urea production, a byproduct of ammonia metabolism, fibrinogen, and A1AT (FIG. 3C, FIG. 3D, FIG. 3E, respectively) seen at the end of the initial 24-hour culture period on day one by the humanized BELs, suggest that human hepatocytes are better suited as a therapy model.

Patency is a critical factor in providing therapy windows long enough to support a patient in acute liver failure. To determine patency in both human and porcine BELs, an in-house blood loop was developed that allowed for oxygenated and temperature-controlled blood to flow through the BEL, while monitoring in-line pressure as a metric of blood flow through the organ (FIG. 3B). When compared to porcine BELs, human BELs had lower pressures over the 180-minute assay remaining within normal physiological parameters (FIG. 3C).

BELs seeded with PHLCs demonstrated higher urea, fibrinogen, and A1AT production than BELs seeded with PPLCs one-day after seeding (FIG. 3D-FIG. 3F). All BELs tested remained patent and supported continuous flow of oxygenated and temperature-controlled whole porcine blood through a 3 hour blood loop assay (FIG. 3G-FIG. 3H). When compared to BELs seeded with PPLCs, BELs seeded with PHLCs had lower pressures over the 180 minute assay.

### Example 16: Anti-Viral Treated and Engineered Livers have Prolonged Stability

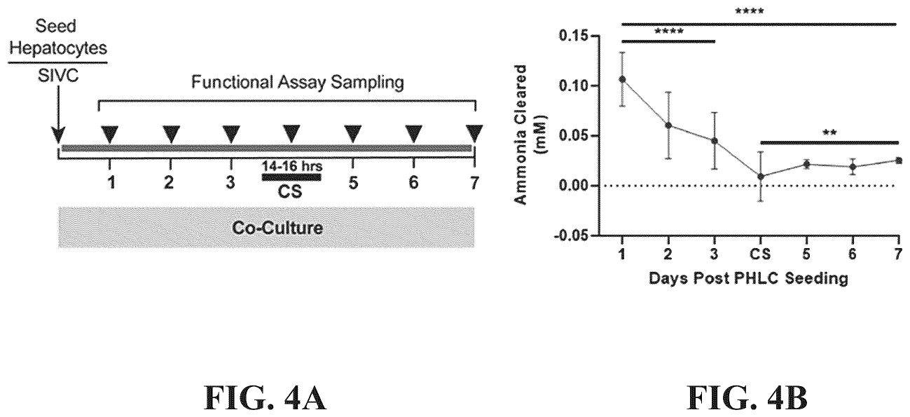

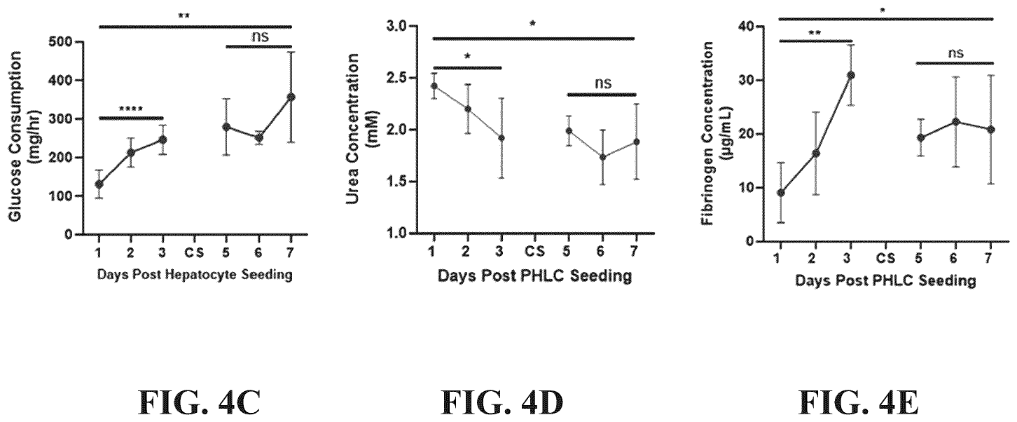

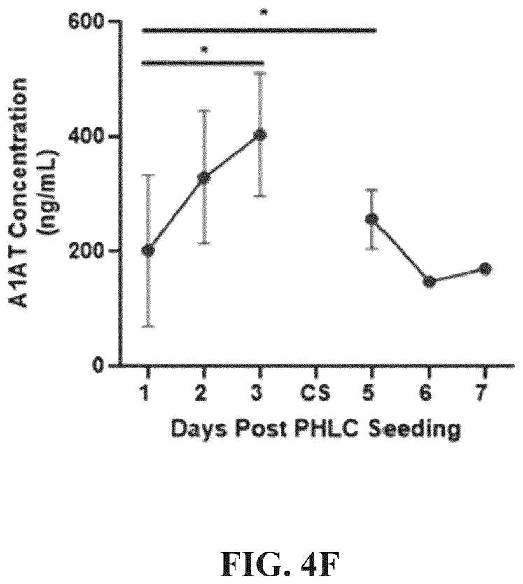

Treatment of ALF patients in a clinical setting requires overcoming some logistical hurdles to provide effective treatment. A bioengineered liver will require sustained function to accommodate patient identification as well as the ability to sustain functionality after transportation. To determine the functional stability of the BEL for extended time periods, BELs were cultured for 72 hours after PHLC seeding with daily sampling and ammonia clearance assessment. BELs were then subjected to a 14-16 hr cold storage period, re-installed into the bioreactor, and cultured for an additional 72 hrs, again with daily sampling and ammonia clearance assessment (FIG. 4A). BELs demonstrated the ability to metabolize ammonia over the entire 72 hr culture period. Clearance rates dropped immediately after the cold storage (within 10 minutes) but stabilized and remained consistent after 24 hrs of bioreactor culture (FIG. 4B). Further evidence of ammonia metabolism is supported by urea accumulation in the bioreactor media at each timepoint measured (FIG. 4C). BELs also demonstrated protein synthesis capability through the production of both fibrinogen and A1AT. Concentrations of both proteins increased throughout the 72 hr culture period post PHLC seeding. Measured levels of both were lower after the cold storage period but were consistent with concentrations recorded 1-2 days after PHLC seeding (FIG. 4D, FIG. 4E). Measured levels of these proteins were lower after the cold storage period but were consistent with concentrations recorded 1-2 days after PHLC seeding (FIG. 4E, FIG. 4F).

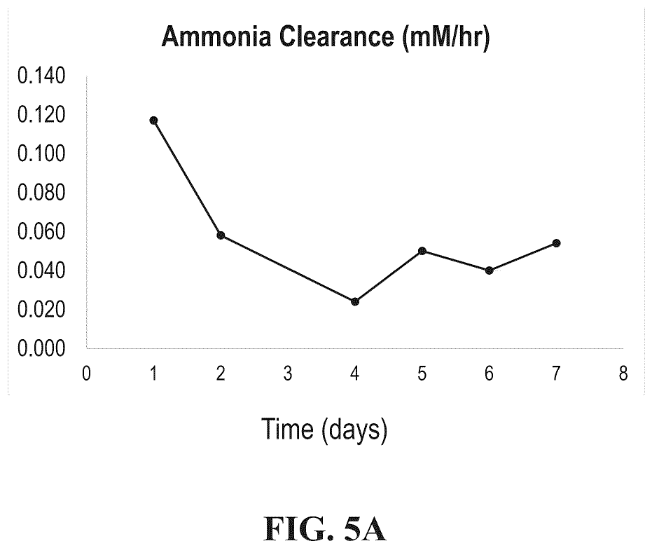

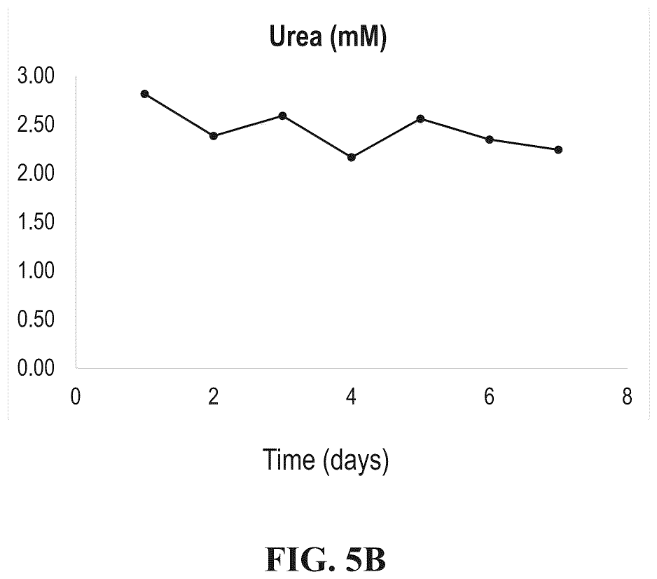

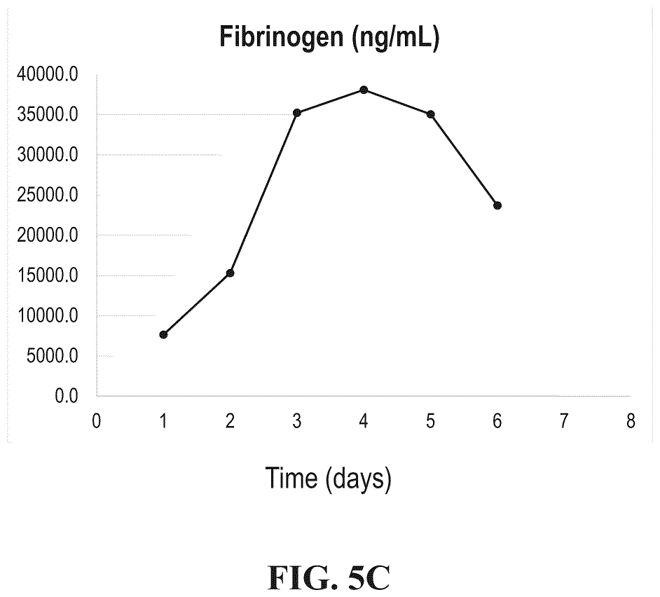

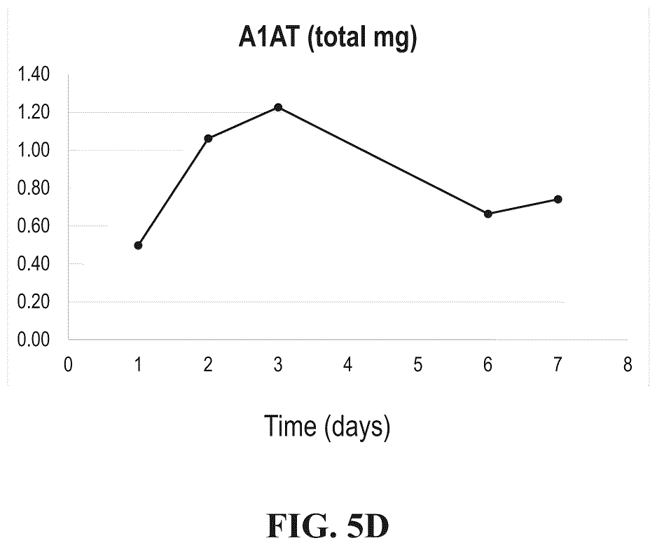

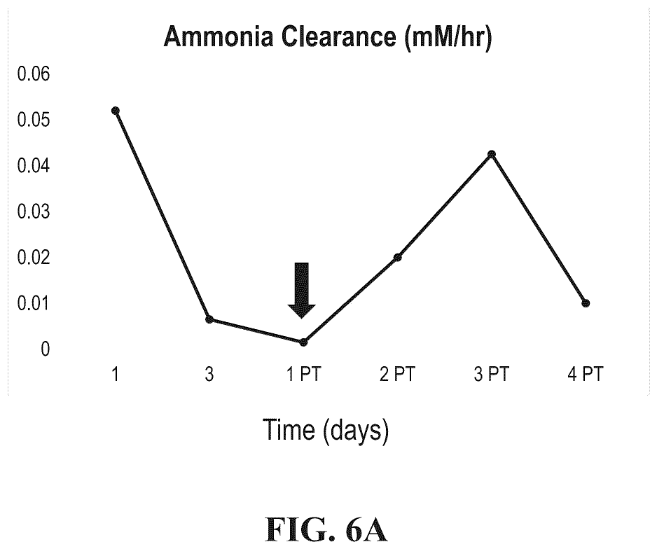

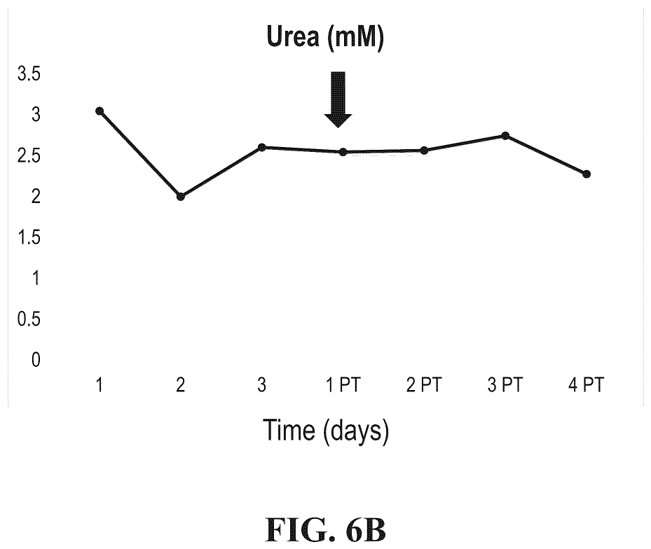

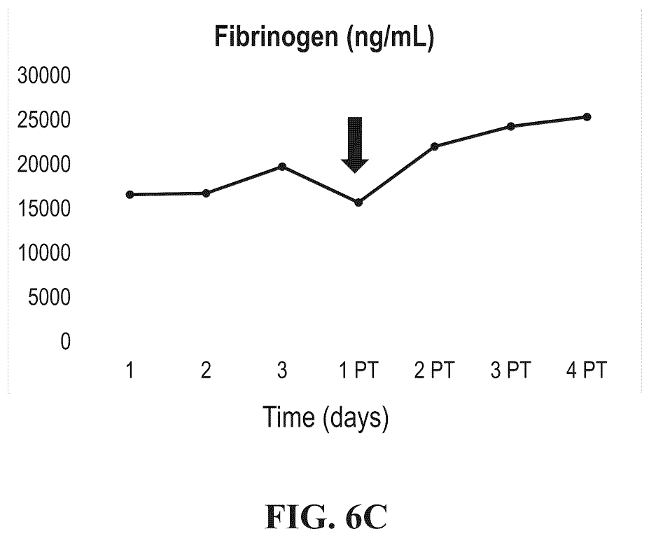

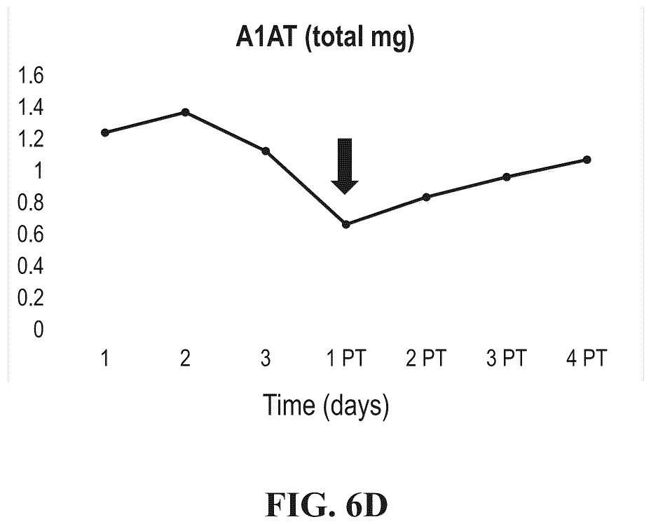

To determine the functional stability of the BEL for extended time periods, BELs were cultured for up 5-7 d after PHLC seeding with daily sampling and ammonia clearance assessments. BELs were then subjected to a 14-16 h cold storage period, re-installed into the bioreactor, and cultured for up to an additional 5 h, again with daily sampling and ammonia clearance assessments. BELs demonstrated the ability to metabolize ammonia up to at least 7-d culture period (FIG. 5A-FIG. 5D). Clearance rates dropped immediately after the cold storage but stabilized and remained consistent after 24 h of bioreactor culture (FIG. 6A-FIG. 6D).

Daily glucose consumption, measured over the entirety of the culture period, steadily increased prior to the culture period and stabilized after the cold storage hold, signifying that the cells of the BEL remained viable and metabolically active. Further evidence of ammonia metabolism is supported by the presence of urea in the bioreactor media at each timepoint measured. BELs also synthesized proteins demonstrated by the production of both fibrinogen and A1AT. Concentrations of both proteins increased throughout the culture period post PHLC seeding as shown in Table 4 below.

TABLE 4
Protein Concentration Over 7 Day Culture of BELs.

| Culture | AC | Urea | A1AT | Fibrinogen |
|---|---|---|---|---|
| Day | (mM) | (mM) | (total mg) | (ng/mL) |
| 1 | 0.117 | 2.81 | 0.50 | 7609.9 |
| 2 | 0.058 | 2.38 | 1.06 | 15268.3 |
| 3 | NT | 2.59 | 1.22 | 35180.6 |
| 4 | 0.024 | 2.16 | NT | 38034.2 |
| 5 | 0.050 | 2.56 | NT | 34998.0 |
| 6 | 0.040 | 2.34 | 0.66 | 23672.0 |
| 7 | 0.054 | 2.24 | 0.74 | NT |

NT = Not tested

### Example 17: Manufacturing Organs with an Intact Exterior Surface Using Freezing and Irradiation Processes

One of the hurdles to e-beam or gamma sterilizing tissues, whole or decellularized, is maintaining the shape of the organ throughout the process. Crosslinking can cause organs to maintain the shape in which they were processed, thus causing problems with shape and perfusion at recellularization. The inventors have developed a method to freeze organs prior to e-beam processing. This allows them to be orientated in, and maintain, a specific shape throughout processing.

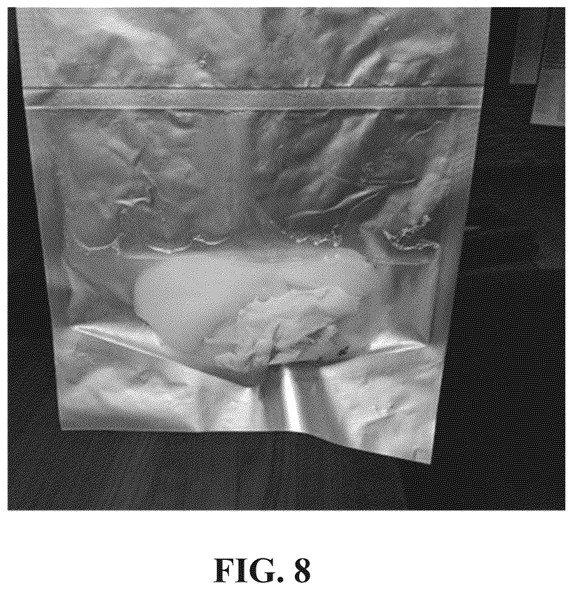

Ideally, decellularized organs would be e-beam sterilized prior to recellularization to ensure viral inactivation is achieved within the process. Earlier attempts to e-beam sterilize at this point lead to issues with orientation and shape. Because decellularized organs deflate during shipping they were unable to maintain their shape while suspended in solution (FIG. 8). This led to issues with reperfusion during recellularization as organs never fully reinflated. Organs that were fully inflated right before e-beam processing were able to fully reinflate, but processing this way was not logistically feasible.

To maintain the shape of the organ, the inventors froze whole porcine organs and e-beam processed while frozen. This gives the ability to maintain the full shape and orientation of the organ during both shipping and processing. Once decellularized, the organs were able to be fully reinflated for recellularization and performed similar to a non-e-beam processed matrix.

Frozen processing can be used to maintain shape of various organs and tissues during e-beam and gamma processing. The process also applies to both native/whole tissues or previously decellularized tissues.

A method of manufacturing organs with an intact exterior surface using freezing and irradiation processes is further provided below.

Whole porcine livers are harvested and collected. These non-sterile organs are placed inside of a vacuum seal bag and arranged similar to the respective organ's native orientation. All organ vessels are additionally placed in their native orientation. The vacuum seal bag is then installed inside of a vacuum sealer. The vacuum sealer is then operated to create a vacuum inside of the bag containing the organ and then sealed to hold that vacuum. The vacuum within the bag helps maintain native organ structure and promote even distribution of irradiation treatment. The vacuum sealed bag containing the organ is then placed inside of a −20 degree Celsius freezer. These packaged, frozen whole organs can be stored for a length of time prior to being sent for electron beam or gamma processing. To send for sterilization, organs are packaged frozen which can include but limited to whole organ inside of a cushioned, insulated box for overnight shipment on dry ice to the electron beam irradiation facility. The organ is then irradiated and returned to the end user.

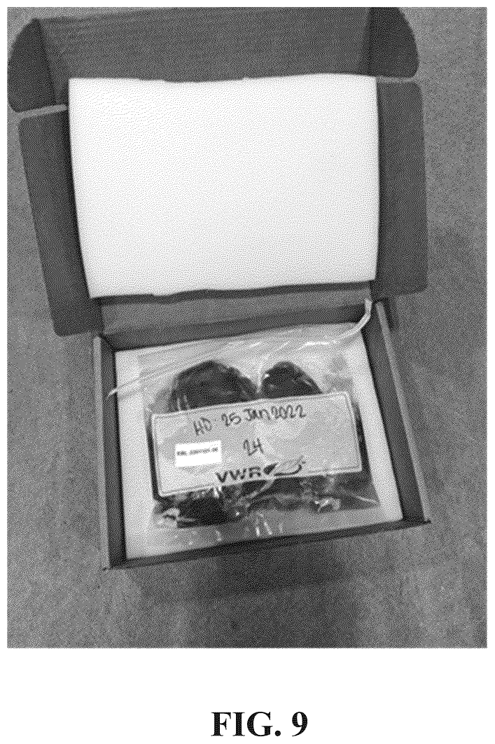

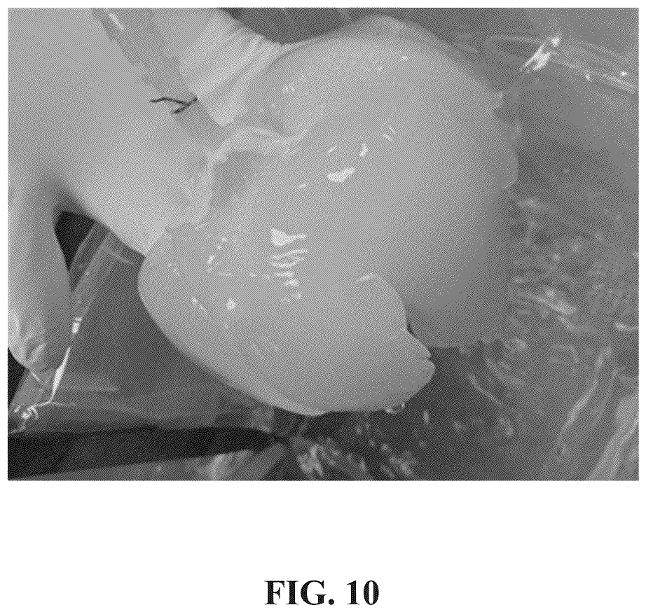

Frozen livers were sent for e-beam processing and two separate recellularization experiments have shown confidence in e-beam processed livers for recellularization. FIG. 9 shows a photograph of the frozen livers sent for e-beam processing, positioned flat for more effective processing and orientation of both the organ and the vessels. FIG. 10 shows a perfusion-decellularized porcine liver.

Original results from e-beam processed decellularized liver grafts showed frequent deformation. These areas generally showed as indentations that could not be fully reinflated and caused blockages to perfusion in certain areas. FIG. 8 shows an e-beam processed graft with damage to lobe 3 and 4.

The major advantage of the methods provided herein are the ability to stabilize the tissue in the desired shape and orientation during shipping and processing, in addition to inactivating viruses. A fresh, non-frozen, porcine organ can be stabilized but is logistically challenging to process as the tissue cannot be preserved as long as frozen tissue. A decellularized organ is easier to preserve but much more difficult to stabilize. Adventitious agents pose one of the largest hurdles to biological organ engineering. Processes must be able to demonstrate the ability to inactivate or remove bacterial, fungal, and viral contamination of the organ. Here the inventors have developed a method to e-beam process whole organs, prior to perfusion decellularization and recellularization, without negatively impacting downstream processes. The e-beam processing step allows for viral inactivation as well as a fungal and bacterial load reduction to occur prior to the start of perfusion decellularization and recellularization.

### Example 18: Methods of Perfusion Decellularizing an Organ

Perfusion decellularization is a process that requires a balance between aggressive disinfection and gentle decellularization to preserve the integrity of the extracellular matrix to allow for effective downstream recellularization. Disinfection steps can prove harmful to the matrix in a way that does not allow for recellularization, and a lack of adequate disinfection can result in contamination that disrupts recellularization and ultimately poses a risk to the patient. Furthermore, viral infections pose a patient threat in perfusion decellularization sourced engineered organs. E-beam processing creates a viral inactivation step that is critical to the safety of the final organ.

E-beam processing prior to decellularization also provides a means to mitigate contamination on the organ as it starts the decellularization process. This is a major benefit, as sourcing sterile organs is difficult to do in an abattoir and often requires the procurement to occur in an operating room setting. E-beam also provides a method for the control of endotoxin in the final product. While contaminated organs may be disinfected during the decellularization process, endotoxin accumulation can cause a major problem in the time between procurement and disinfection. The contamination reduction from the e-beam processing step mitigates endotoxin levels that pose a threat to the patient (if sterilization occurs after the decellularization process).

Whole, porcine organs such as a liver or kidney are collected from donor animals. These non-sterile organs are placed inside of a vacuum seal bag and arranged within the respective organ's native orientation. All organ vessels are additionally placed in their native orientation. The vacuum seal bag is then installed inside of a vacuum sealer. The vacuum sealer is then operated to create a vacuum inside of the bag containing the organ and then sealed to hold that vacuum. The vacuum within the bag helps maintain native organ structure and promote even distribution of irradiation treatment. The vacuum sealed bag containing the organ is then placed inside of a −20 degrees Celsius freezer. By freezing the tissue, it can further maintain the liver orientation and claim to pause and prevent decomposition of the native tissue. These packaged, frozen whole organs can be stored for a length of time prior to being sent for electron beam irradiation. To send for sterilization, the inventors package the frozen, whole organ inside of a cushioned, insulated box for overnight shipment on dry ice to the irradiation facility. The irradiation facility then receives the organs and individually doses each organ with a pre-determined dose of irradiation per inventors' specification. During process development, electron beam irradiation dose mapping was completed on several livers to ensure proper irradiation exposure across the entirety of the liver graft. The whole organs are then sent back to the inventors on dry ice and received frozen into their possession. These sterilized organs can then be placed into the freezer for a period before being thawed for decellularization.

In-process whole organ sterilization that does not have a negative downstream impact on decellularization/recellularization was performed. Table 5 shows the assay conditions for each treatment with a low dose or high dose E-beam relative to a control group.

TABLE 5
Treatment Conditions.

| Group | Low Dose E-Beam | High Dose E-Beam | Control |
|---|---|---|---|
| Treatment | 10 kGy E-beam | 20 kGy E-beam | No E-beam Dose* |
| Number | 4 | 4 | 2 |
| Cell Line | Lonza 621 | Lonza 621 | Lonza 621 |
| Bioreactor | 2.0 C | 2.0 C | 2.0 C |
| Seeding | 200M PV/ | 200M PV/ | 200M PV/ |
| | 200M HV | 200M HV | 200M HV |
| Media Scheme | Every other | Every other | Every other |
| | day media change | day media change | day media change |
| Final Assay | 1 hr Blood Loops (x3) | 1 hr Blood Loops (x3) | 1 hr Blood Loops (x2) |
| | Histology (x1) | Histology (x1) | |

*control grafts were not subjected to the exact same handling as the e-beam grafts.

Recellularized livers showed favorable results for e-beam processing. Five grafts (three high dose E-beam and two low dose E-beam treated) peaked higher than the control grafts. Additionally, there was little difference between the high dose (20 kGy) and low dose (10 kGy) groups (FIG. 11).

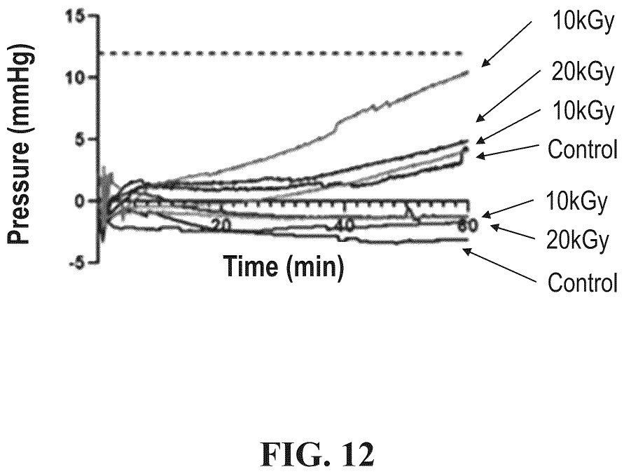

Patency results were also favorable, with all grafts passing the assay and being deemed patent. There were also no noticeable trends among pressures during the test. All groups had at least one graft finish above 4 mmHg, and all groups had one or more grafts finish with a negative pressure, including two grafts from the high dose group (FIG. 12).

Overall, the data of Example 18 shows that e-beam processing was viable for whole organ sterilization and viral inactivation in decellularized livers.

### Example 19: Consistent Manufacturing of Functional Recelluarlized Livers

The data presented herein establishes a defined and consistent manufacturing process to create functional BELs, composed entirely of human cell types, as a bridge therapy for acute liver failure. The decellularization process provided herein generates extracellular matrices that are free from xenotropic virus transmission, and without any remaining residual Triton, SDS, or porcine DNA that could present potential complications for ALF patients. The seeding methods provided herein enable coverage of the vasculature by the HUVECs and infiltration of the PHLCs into the parenchyma. The bioreactor maintains BEL culture parameters to facilitate consistent cellular growth resulting in six days of positive functional output.

One of the underlying concerns with xeno-materials for patient therapy is the transmission of foreign material, potentially those that can ignite an immune response in an already critically ill patient. As the porcine-based material is the decellularized liver scaffold, this was addressed by employing a viral inactivation strategy that utilized both E-Beam irradiation and a PAA treatment, orthogonal modalities that together successfully clear viruses. The model viruses used in this study were selected for their unique characteristics based upon their genome, envelope, and shape. The ability of this treatment to inactivate or remove these viruses builds confidence in the approach as a valid manufacturing method to remove several varieties of viruses that might negatively impact the patient. Additionally, removal of toxic residuals as a byproduct of the decellularization process was verified and confirmed the sterility of the recellularization process signifying that the BELs described herein are safe for patient therapy.

Previous assays on porcine hepatocytes for the development of BELs showed the risks of xenotropic virus transmission and the possible immunological response of the human patient towards porcine cells limits the utility of that strategy to provide therapy for ALF. PHLCs not only circumvent these concerns, but provide increased function throughout the culture period when compared to the porcine hepatocytes. While some BELs seeded with porcine hepatocytes are able to clear ammonia at a similar level on day one, the inability of porcine hepatocytes to retain function by culture day three makes providing therapy to a patient impractical, since several logistical hurdles have to be considered in order to transport the organ to a patient in need. Furthermore, the low production of fibrinogen and A1AT point to an inferior material that does not have the metabolic capability of PHLCs.

After cold storage hold to mimic the transport process to a patient, a drop in ammonia clearance, and fibrinogen, A1AT, and urea production were observed briefly. This is not unexpected as cold ischemic time is well known to have a negative impact on tissue metabolism. However, when compared to the first day after cold storage BEL function largely remains steady for the duration of the culture period. The ammonia clearance rates, urea and fibrinogen production collected post cold storage remained stable for seventy hours beyond the transport period which opens the door for BEL as a potent therapy for ALF patients. Future studies will examine the ability of BELs, generated through defined and consistent manufacturing process, to provide patient therapies in a clinical setting.

Acute liver failure is a rapidly devasting disease. The high demand and long wait times for a liver transplant puts a patient at risk. The extracorporeal bioengineered liver can be used to support patients either in recovery or as a bridge to transplant. The manufacturing process provided herein is capable of generating bioengineered liver grafts that are able to adhere to the safety standards required for patient treatment. The ability of the bioengineered livers to exhibit function for an extended period of time, demonstrate activity after a cold storage transportation, and provide extended function in a prolonged therapy windowpoints to the potential to provide effective therapy. The results of the assays in Examples 1-18 above, show that the extracorporeal bioengineered liver is a clinically translatable product for the treatment of ALF.

What is claimed is:

1. A method of making an at least partially recellularized liver composition, the method comprising:
(a) treating a non-human animal liver with an anti-viral treatment;
(b) perfusion decellularizing the non-human animal liver to obtain a decellularized extracellular matrix;
(c) contacting the decellularized extracellular matrix with a cell composition comprising a population of human liver cells to form an at least partially recellularized liver composition.

2. The method of claim 1, wherein the anti-viral treatment comprises irradiation of the non-human animal liver with an electron beam (E-beam).

3. The method of claim 2, wherein the irradiation comprises exposing the non-human animal liver to an electron beam dose that is from about 2 kGy to about 50 kGy.

4. The method of claim 1, further comprising contacting the decellularized extracellular matrix with at least one of a peroxy acid or hydrogen peroxide.

5. The method of claim 1, wherein the anti-viral treatment comprises irradiation of the non-human animal liver with an electron beam (E-beam), and wherein the method further comprises contacting the decellularized extracellular matrix with at least one of a peroxy acid or hydrogen peroxide.

6. The method of claim 1, further comprising contacting the decellularized extracellular matrix with an additional cell composition comprising a population of human vascular endothelial cells (HUVECs).

7. The method of claim 1, wherein the cell composition comprises both a population of human liver cells and a population of HUVECs.

8. The method of claim 1, wherein the population of human liver cells are primary human liver cells or in vitro-differentiated human liver cells.

9. The method of claim 1, wherein the population of human liver cells are contacted with a protease prior to contacting the decellularized extracellular matrix with the cell composition in step (c).

10. The method of claim 1, wherein the at least partially recellularized liver is characterized as having an increase in a level of ammonia clearance relative to a population of liver cells that are not engrafted onto a decellularized extracellular matrix.

11. The method of claim 1, wherein the non-human animal liver is from a non-human mammal.

12. The method of claim 1, wherein the non-human animal liver is frozen prior to the anti-viral treatment.

13. A method of making an at least partially recellularized liver composition, the method comprising:
(a) treating a non-human animal liver with an anti-viral treatment;
(b) perfusion decellularizing the non-human animal liver to obtain a decellularized extracellular matrix;
(c) contacting the decellularized extracellular matrix with a first cell composition comprising a population of human vascular endothelial cells; and
(d) contacting the decellularized extracellular matrix with a second cell composition comprising a population of human liver cells to form an at least partially recellularized liver composition.

14. The method of claim 13, wherein the decellularized extracellular matrix is contacted with the second cell compositions when the first cell composition is characterized as having a glucose consumption rate of at least about 30 mg/hr.

15. The method of claim 13, wherein the decellularized extracellular matrix is contacted with the second cell composition at least 10 days after contacting the decellularized extracellular matrix with the first cell composition.

16. The method of claim 13, wherein the at least partially recellularized liver composition is characterized as having an increase in a level of ammonia clearance relative to a population of liver cells that are not in the form of an at least partially recellularized liver composition.

17. The method of claim 13, wherein the anti-viral treatment comprises irradiation of the decellularized extracellular matrix with an electron beam (E-beam).

18. The method of claim 17, wherein the irradiation comprises exposing the non-human animal liver to an electron beam dose that is from about 2 kGy to about 50 kGy.

19. The method of claim 13, further comprising contacting the decellularized extracellular matrix with a peroxy acid or hydrogen peroxide.

20. The method of claim 13, wherein the population of human liver cells are primary human liver cells or in vitro-differentiated human liver cells.

21. The method of claim 13, wherein the non-human animal liver is frozen prior to the anti-viral treatment.

22. A composition comprising an at least partially recellularized liver composition produced by the method of claim 1.

23. A composition comprising an at least partially recellularized liver composition produced by the method of claim 13.

24. An at least partially recellularized liver comprising:
(a) a microbial particle diminished, perfusion-decellularized porcine extracellular matrix;
(b) a population of human endothelial cells and a population of human liver cells engrafted onto the porcine extracellular matrix,
wherein the at least partially recellularized liver has an increase in ammonia clearance relative to a population of porcine liver cells engrafted onto a porcine extracellular matrix.

25. An ex-vivo method of treating a liver disease in a subject, the method comprising:
producing a blood circuit, wherein the blood circuit comprises blood from the subject in fluid communication with an at least partially recellularized liver of claim 24, wherein the at least partially recellularized liver filters blood from the subject and clears ammonia, thereby treating the liver disease in the subject.
